(19) United States
(12) Patent Application Publication
(10) Pub. No.: US 2023/0293050 A1
(43) Pub. Date: Sep. 21, 2023

(54) WEARABLE AMBULATORY SENSORS FOR MEASUREMENT OF BREATHING MOTIONS

(71) Applicant: Regents of the University of Minnesota, Minneapolis, MN (US)

(72) Inventors: Rajesh Rajamani, Minneapolis, MN (US); Paolo Pianosi, Minneapolis, MN (US); Gregory W. Johnson, Minneapolis, MN (US)

(73) Assignee: Regents of the University of Minnesota, Minneapolis, MN (US)

(21) Appl. No.: 18/017,610

(22) PCT Filed: Jul. 27, 2021
(86) PCT No.: PCT/US2021/070963

§ 371 Date: Jan. 23, 2023

Related U.S. Application Data
(60) Provisional application No. 63/057,494, filed on Jul. 28, 2020.

(51) Int. Cl. A61B 5/113; A61B 5/08; A61B 5/00

(52) U.S. Cl. CPC A61B 5/1135; A61B 5/0816; A61B 5/6823; A61B 5/6833; A61B 5/725; A61B 2562/0219

(57) ABSTRACT

An example wearable respiratory motion sensor system includes a plurality of inertial measurement units (IMUs) to be positioned on a subject and generate accelerometer and gyroscope signals. The wearable respiratory motion sensor system also includes a processor to compute three-dimensional displacements of a rib cage and an abdomen of the subject based on the generated accelerometer and gyroscope signals.

### CROSS-REFERENCE TO RELATED APPLICATIONS

This 35 U.S.C. § 371 National Phase application claims priority to International Application No. PCT/US2021/070963, filed Jul. 27, 2021, which claims the benefit of U.S. Provisional Patent Application No. 63/057,494, filed Jul. 28, 2020; which are both incorporated herein by reference in their entirety.

### BACKGROUND

Accurate measurement of pulmonary ventilation or breathing may involve the use of devices such as masks or mouthpieces coupled to the airway opening. These devices may be both encumbering and invasive, and thus not well-suited for continuous or ambulatory measurements.

As an alternative, respiratory inductance plethysmography (RIP) devices that sense respiratory excursions at the body surface may be used to measure pulmonary ventilation. A respiratory inductance plethysmograph may consist of two sinusoid wire coils insulated and placed within two lightweight elastic and adhesive bands. The transducer bands are placed around the rib cage under the armpits and around the abdomen at the level of the umbilicus (belly button). They are connected to an oscillator and subsequent frequency demodulation electronics to obtain digital waveforms. During inspiration, the cross-sectional area of the rib cage and abdomen increases altering the self-inductance of the coils and the frequency of their oscillation, with the increase in cross-sectional area proportional to lung volumes. The electronics convert this change in frequency to a digital respiration waveform where the amplitude of the waveform is proportional to the inspired breath volume.

While RIP can provide a measure of chest displacement (which can also be used to calculate tidal volume), it may not be extended to measure displacements along three axes, so detailed three-dimensional measurements of chest or abdominal motion measurements may not be performed with this technique.

Non-invasive camera-based surface registration methods may be used to register and track breathing motions in a patient's abdomen and thorax. Such systems may be laboratory based and expensive. The patient must lie down and be static for such measurement systems to be utilized. They may not be utilized for home-based or ambulatory monitoring.

Accelerometers may be used to measure respiratory movements. However, such methods may estimate either respiratory rate or timing intervals between heartbeats, or angular rates of chest motion and then correlate these values to flow rate measurements, rather than directly measuring actual chest or abdominal displacements.

For these and other reasons, a need exists for the present invention.

### SUMMARY

One example is directed to a wearable respiratory motion sensor system that includes a plurality of inertial measurement units (IMUs) to be positioned on a subject and generate accelerometer and gyroscope signals. The wearable respiratory motion sensor system also includes a processor to compute three-dimensional displacements of a rib cage and an abdomen of the subject based on the generated accelerometer and gyroscope signals.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is a schematic diagram illustrating a human subject with a wearable breathing motion measurement system according to one example.

FIG. 2 is a diagram illustrating example data showing significant drift when accelerations are double-integrated.

FIG. 3 is a diagram illustrating the performance of a high pass filter in removing drift from double-integrated accelerometer signals.

FIG. 4 is a diagram illustrating a wearable strap that includes an electromagnet on the strap, in addition to a sensor board with a 3-axis accelerometer, a single-axis or multiple-axis (e.g., 3-axis) magnetometer, and a wireless transceiver.

FIG. 5 is a diagram illustrating a least mean squares (LMS) adaptive feedforward filter based on a reference signal.

FIG. 6 is a diagram illustrating the use of an additional accelerometer on a user's waist to measure a reference signal related to walking by the subject.

FIG. 7 is a diagram illustrating an adaptive feedforward method using least mean squares to remove the influence of walking by using an accelerometer on the waist or other body location to measure the walking of a human subject and removing the signal due to walking.

FIG. 8 is a diagram illustrating an arrangement of IMUs on a human subject to accommodate low sensitivities at very low frequencies, and to detect differential movement of the thoracic ribcage, abdominal ribcage, and abdomen.

FIGS. 9A-9C are diagrams illustrating one example implementation of a system with wearable ambulatory sensors for measurement of breathing motions.

FIG. 10 is a diagram illustrating a Kalman smoother overview according to one example.

FIG. 11 is a diagram illustrating how the Kalman smoother solves a lag issue.

FIGS. 12-26 are diagrams illustrating graphs of measurements made by the system shown in FIG. 9.

FIG. 27 is a diagram illustrating a linear FIR filter model that may be used to estimate tidal volume according to one example.

FIG. 28 is a diagram illustrating an equation for calculating tidal volume according to one example.

FIG. 29 is a diagram illustrating model fitting using a training set batch for the linear FIR filter model shown in FIG. 14 according to one example.

FIG. 30 is a block diagram illustrating a wearable respiratory motion sensor system according to one example.

FIGS. 31A and 31B are diagrams illustrating experimental results for detection of paradoxical thoracoabdominal displacements.

FIGS. 32A-32D are diagrams illustrating a system for estimating tidal volume according to an example.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

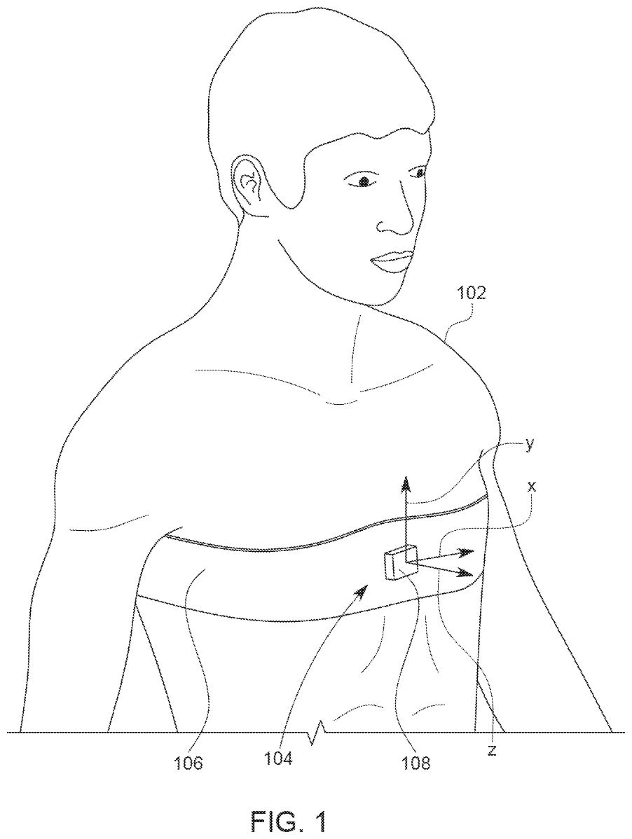

Some examples disclosed herein are directed to a convenient wearable sensor system for wireless measurement of chest and abdominal motions during breathing. The system includes one or more wearable straps or adhesive pads with sensors and wireless electronics embedded on each strap or pad. Each strap or pad measures oscillatory breathing motions along three mutually perpendicular axes (for example, the axes x, y and z shown in FIG. 1). FIG. 1 is a schematic diagram illustrating a human subject 102 with a wearable breathing motion measurement system 104 according to one example. The measurement system 104 consists of a wearable strap 106 with an embedded sensor board 108 which measures and wirelessly transmits displacements along three mutually perpendicular axes. In other examples, an adhesive pad may be used in place of the wearable strap 106. The components on each strap 106 (or adhesive pad) according to some examples include an inertial measurement unit (e.g., containing at least a 3-axis accelerometer and a 3-axis gyroscope), a wireless transceiver and a microprocessor containing estimation methods. More than one strap 106 (for example, one on the chest and one on the abdomen) or adhesive pad may be utilized and the relative phase of the corresponding displacements measured by the two, three, or more straps (or adhesive pads) may be utilized for interpreting muscular action during breathing. In addition, additional components such as an electromagnet and a magnetic sensor may be incorporated in some examples of the sensor, as described further later in this disclosure. Another feature of this disclosure is the computation of relative motions and phase differences between the breathing motions of the thoracic vs abdominal ribcages, and vs the abdomen in order to interpret the detailed muscular actions of a human subject during breathing. Such interpretations may be used to help diagnose and monitor the neuromuscular health of the respiratory muscles of a human subject.

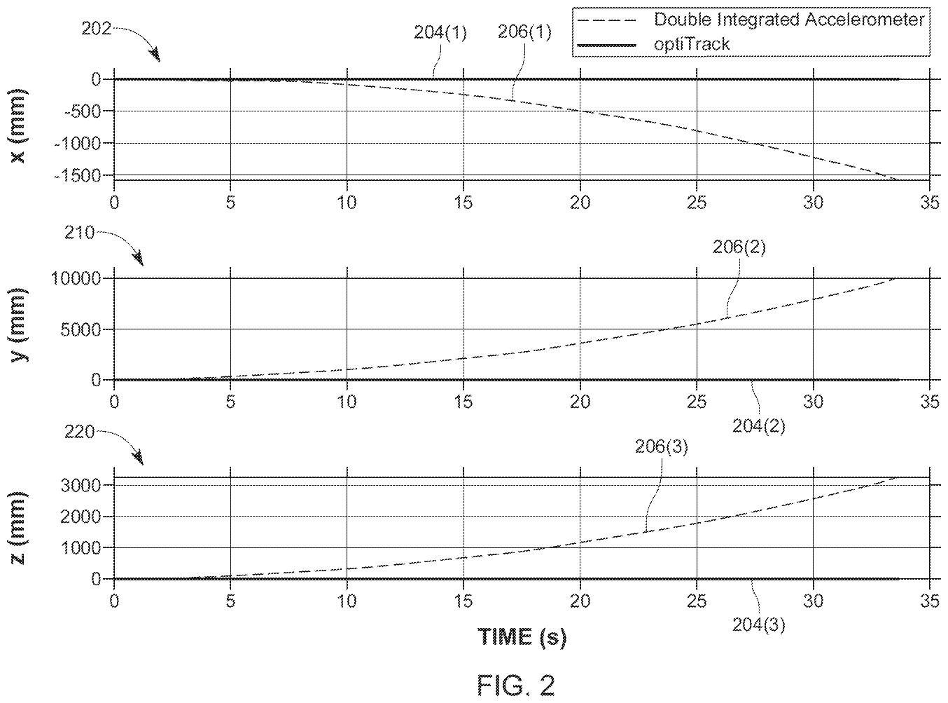

One issue addressed by some examples disclosed herein is drift from accelerometer integration. A 3-axis accelerometer on the strap 106 (or adhesive pad) measures accelerations along three mutually perpendicular axes. For example, it can measure the variables \ddot{x}, ÿ and \ddot{z} along the three axes x, y, and z shown in FIG. 1 (ignoring gravity components for now). Here z refers to anterior-posterior motion, x refers to circumferential motion or lateral and y refers to upwards-downward motion. While the accelerometer signals and can be double integrated in an attempt to obtain x, y and z, such double integration will invariably lead to drift. FIG. 2 is a diagram illustrating example data showing significant drift when accelerations are double-integrated. FIG. 2 shows how the double integrated accelerometer signals drift significantly with time. The double integrated accelerometer signals 206(1)-206(3) in this figure are the double-integrated values of accelerations along the three axes of the accelerometer. Graph 202 represents acceleration along the x-axis, graph 210 represents acceleration along the y-axis, and graph 220 represents acceleration along the z-axis. This example data was taken by using a 3-axis accelerometer on a wearable chest strap. The double integrated accelerometer signals 206(1)-206(3) are compared to Optitrack signals 204(1)-204(3), respectively. The Optitrack is a laboratory infrared camera system that accurately measures displacements and is used as a reference measurement system with which the accelerometer-based estimates are compared.

The reason an integrated signal drifts is because all measured signals have at least a small bias error and the integration of this bias error causes the result to drift continuously. It is not possible to subtract the bias from the measured signal because the bias value will change ever so slightly with time and will not be a constant. Even if the bias is extremely small, its integration results in a drift which eventually grows to become a very large value. Another way to interpret the drift is by noting that integration is a marginally stable operation and hence a constant bias input to this marginally stable operator results in a growing output from the operator.

Another issue addressed by some examples disclosed herein is errors due to gravity. The accelerometers read not only accelerations due to motion, but also acceleration due to gravity (i.e., a constant value of 9.81 m/s² in the direction of gravity). If the gravity direction was along one of the primary axes of the accelerometer, then the gravity value could be subtracted from the real-time reading along that axis. Alternately, if the orientation of the accelerometer sensor in real-time was known, the components of gravity along the three axes could be calculated based on the known orientation, and these components could be subtracted in real-time from their respective readings. So one challenge is in estimating the real-time orientation of the axes of the sensor. The subject wearing the instrumented strap (or adhesive pad) may bend during breathing. For example, it is common for a person to bend synchronously with breathing while taking deep breaths. Due to these changes in orientation of the back (and thus of the sensor), the gravity component that influences the readings along the three axes keeps changing. This can lead to significant errors in estimated chest or abdominal displacements.

Another issue addressed by some examples disclosed herein is errors due to a subject's physical motion. If the subject wearing the instrumented strap (or adhesive pad) walks, runs, climbs, bends or otherwise physically moves, then the accelerometers on the strap (or adhesive pad) will measure the accelerations corresponding to those movements, in addition to measuring the accelerations due to chest or abdominal respiratory expansion. Hence the chest expansions inferred from the accelerometers will have significant error if the measurements are made while the subject is moving.

Another issue addressed by some examples disclosed herein is errors at very low frequencies. When the subject is breathing at very low frequencies (e.g., below 0.2 Hz, which can occur during sleep in adults), the accelerometer readings are extremely small due to the very low frequencies involved. For example, if the x-axis displacement is given by the following Equation I:

x_{displacement}=x_{Q} sin(ωt) Equation I

Then the corresponding acceleration is given by the following Equation II:

aₓ=\ddot{x}_{displacement}=−x_{Q}ω² sin(ωt) Equation II

Due to the multiplication by cot, measured accelerations become extremely small at low frequencies and hence the sensitivity of the measurement to displacement is very low.

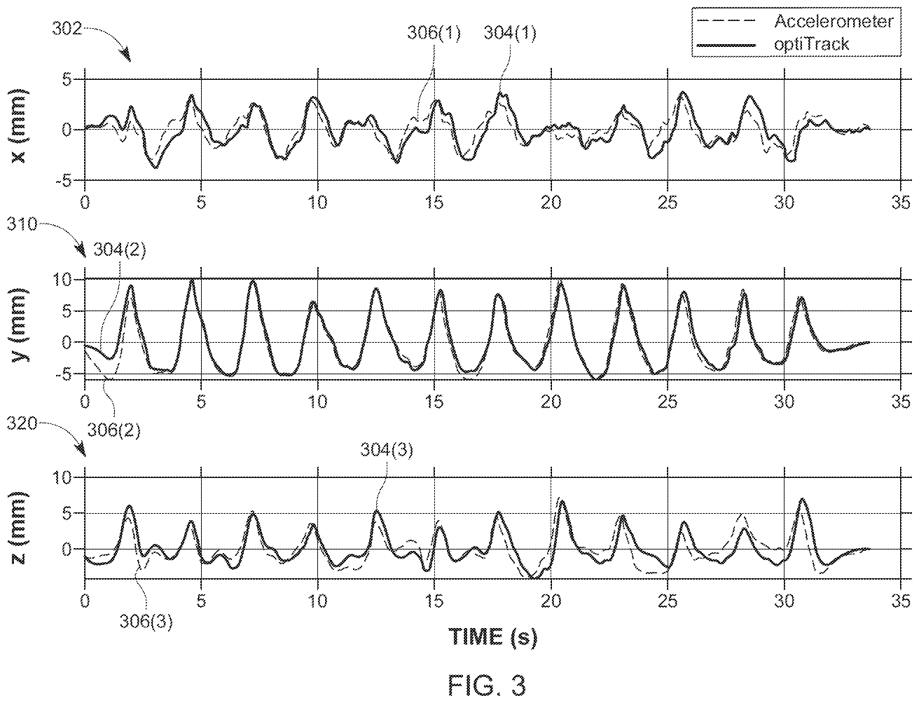

Some examples disclosed herein address the issues identified above in the following manner. High pass filters may be used to remove the drift, if the subject is stationary. A high pass filter can remove the drift in the double-integrated accelerometer signals, if it is properly designed. Since breathing frequency typically varies between 0.1 and 0.8 Hz, a high pass filter should be designed with a corner frequency much smaller than this breathing frequency. A corner frequency of 0.01 Hz is therefore used in some examples, since it is at least 10 time smaller than the lowest expected breathing frequency. This ensures that the components of the signal removed by the high pass filter do not introduce any errors into the chest/abdomen expansion measurement. FIG. 3 is a diagram illustrating the performance of a high pass filter in removing drift from double-integrated accelerometer signals 306(1)-306(3). Graph 302 represents signals along the x-axis, graph 310 represents signals along the y-axis, and graph 320 represents signals along the z-axis. It can be seen that the same data from FIG. 2 (which had significant drift) no longer have a drift, after the double-integrated signals 306(1)-306(3) have been high pass filtered. It can also be seen that the double-integrated and high pass filtered signals 306(1)-306(3) accurately match the chest expansions measured by the infrared optiTrack camera system and represented by signals 304(1)-304(3).

While high pass filters are adequate for signal processing when the subject is stationary, they may be inadequate when the sensor orientation changes due to the subject bending or due to additional unrelated dynamic measurements when the subject is moving. For a situation with a varying sensor orientation, the real-time orientation of the inertial measurement unit (IMU) sensor can be estimated in real-time using a combination of the accelerometers and the gyroscopes in the IMU chip. The estimation method utilizes a combination of the dynamic component from gyroscope integration with a static component from accelerometer measurements. Some aspects of real-time sensor orientation are: (1) the need to ensure that the estimated gravity vector satisfies the 1 g magnitude constraint, the need to compensate for gyroscope bias, and the need to use a Kalman smoother instead of a Kalman filter to calculate the orientation angles.

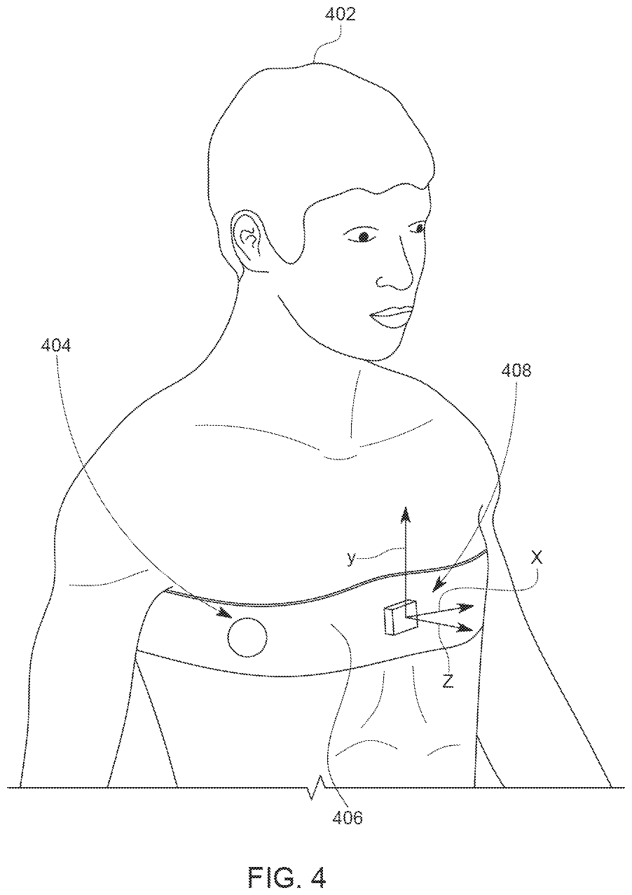

For the case when the subject is moving, some examples disclosed herein may use an additional reference sensor consisting of an electromagnet on the chest strap (or adhesive pad). FIG. 4 is a diagram illustrating a user 402 with a wearable strap 406 that includes an electromagnet 404 on the strap, in addition to a sensor board 408 with a 3-axis accelerometer, a single-axis or multiple-axis (e.g., 3-axis) magnetometer, and a wireless transceiver. In other examples, the electromagnet 404 and the sensor board 408 may be secured to the user 402 with one or more adhesive pads rather than the strap 406. The magnetometer is used to measure the respiratory frequency in real-time by measuring the frequency of variation of the oscillatory magnetic field amplitude. As the strap 406 expands and relaxes in response to respiration, the electromagnet 404 on the strap 406 moves away from and towards the sensor board 408. This change in distance causes a change in the magnetic field read by the magnetometer on the sensor board 408. An oscillatory magnetic field (at a sufficiently high frequency such as 40 Hz) can be created by the electromagnet 404 by providing a corresponding oscillatory current to the electromagnet 404. The electromagnet frequency needs to be significantly higher than the movements of the subject. Since the bending, arm movements, walking and other movements of the subject are expected to be well below 10 Hz, an electromagnet oscillatory frequency significantly higher than 10 Hz can be utilized. By using an oscillatory frequency, the magnetometer signals can be immune to magnetic disturbances from ferromagnetic objects located around the human subject. If magnetic disturbances from ferromagnetic objects are not a source of concern, then a permanent magnet instead of an electromagnet can also be utilized. The magnetic signals at the alternating frequency of the electromagnet 404 will provide a measure of the respiration frequency of the subject in real-time. Knowledge of this respiratory frequency can be used in an adaptive feedforward filter to specifically extract the motions of the strap 406 (or adhesive pad) that correspond only to the respiratory motion (while removing the influence of walking or other motions of the subject).

The overall procedure for finding the respiratory expansion and relaxation displacements according to one example includes the following: (1) Remove the gravity component from the real-time accelerometer readings; (2) double integrate the accelerometer signals; (3) high pass filter the double-integrated signals; and (4) use an adaptive feedforward filter in which the measured real-time respiratory frequency is utilized as a reference signal to extract only the displacements corresponding to respiration (while eliminating movements due to walking, bending or other physical movements of the subject).

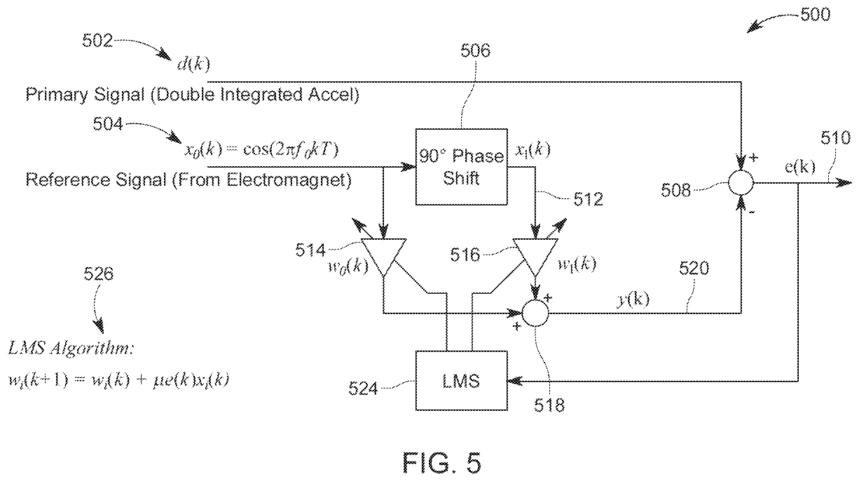

FIG. 5 is a diagram illustrating a least mean squares (LMS) adaptive feedforward filter 500 based on a reference signal 504. The inputs to the filter 500 are a primary signal (e.g., double integrated acceleration) 502 and a reference signal 504. The reference signal 504 (x₀) in this application consists of the respiratory frequency measured by the magnetometer. The filter weights 514 (w₀(k)) and 516 (w₁(k)) may be adjusted in real-time using the adaptive least mean square (LMS) module 524, which may use the equation 526 shown in FIG. 5. Here k is a time-related variable with kT being the actual time, if T is the sampling period utilized. The reference signal 504 is provided to 90 degree phase shifter 506 to produce phase shifted signal 512 (x₁(k)). Weight 514 is applied to the reference signal 504 to produce a first weighted signal, and weight 516 is applied to the phase shifted signal 512 to produce a second weighted signal, and the first and second weighted signals are summed at node 518 to produce estimated signal 520. The estimated signal 520 is subtracted from the primary signal 502 at node 508 to produce filtered signal 510. The filtered signal 510 (e(k)) in this case will contain displacements due to non-respiratory motion, while the estimated signal 520 (y(k)) will contain the displacements due to respiration only.

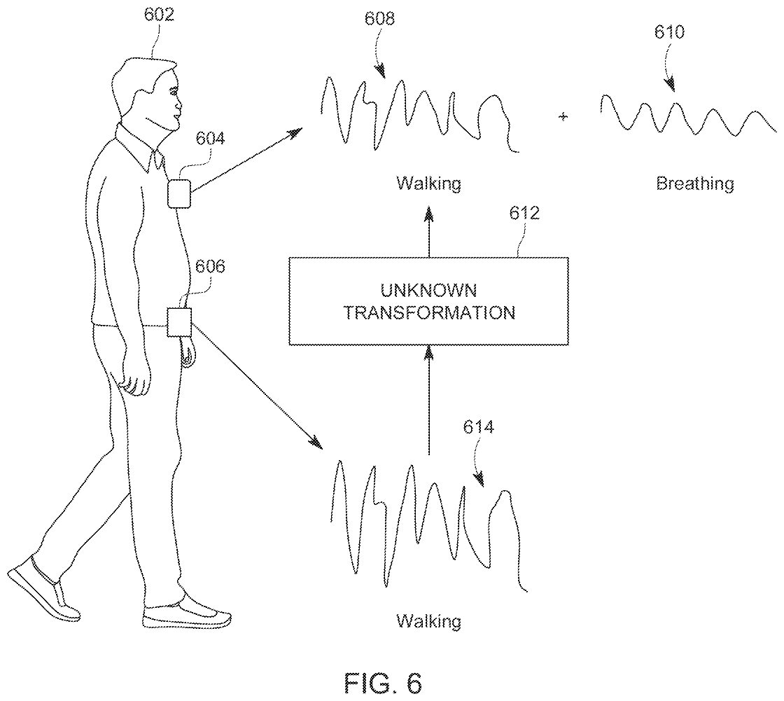

For the case when the subject is moving, some examples disclosed herein may use a reference sensor including an accelerometer on the waist or other part of human body. The method of extracting only the respiration related motion from the wearable strap (or adhesive pad) involves measuring a signal related to the other motions. For example, if it is desired to measure motions related to walking, an accelerometer could be placed on the waist of the human subject. The accelerations due to walking can then be measured and the signal on the wearable strap (or adhesive pad) corresponding to the walking motion can be identified in real-time and removed by using the waist accelerometer signal as a reference signal. FIG. 6 is a diagram illustrating the use of an accelerometer 604 on a chest of a user 602 and an additional accelerometer 606 on the user's waist to measure a reference signal 614 related to walking by the subject. The signal from the accelerometer 604 includes a walking-related signal component 608 and a breathing-related signal component 610. The walking signal 614 from the accelerometer 606 may be provided as a reference signal to a transformation module 612 to identify and remove the walking-related signal component 608 from the signals produced by accelerometer 604. One example of this second method is similar to the first method described above that involves an electromagnet on the chest strap (or adhesive pad), except this second method utilizes a reference signal related to respiratory motion, while the first method utilizes a reference signal related to walking.

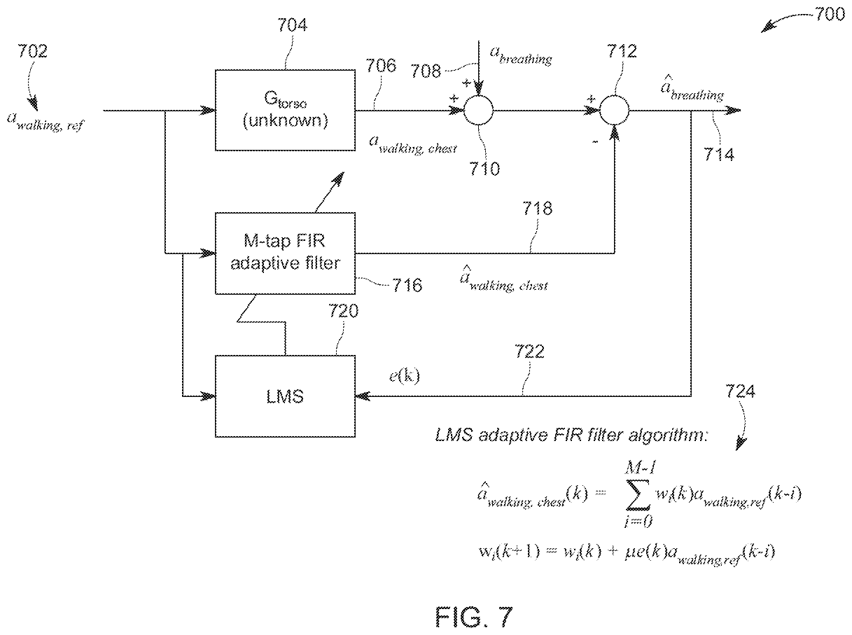

FIG. 7 is a diagram illustrating an adaptive feedforward method 700 using least mean squares to remove the influence of walking by using an accelerometer on the waist or other body location to measure the walking of a human subject and removing the signal due to walking. A walking reference signal 702 is provided to unknown transformation module 704, M-tap Finite Impulse Response (FIR) adaptive filter 716, and least mean squares (LMS) module 720. Module 704 outputs a walking, chest signal 706. Filter 716 outputs a filtered walking, chest signal 718. Node 710 sums the walking, chest signal 706 and a breathing signal 708, and outputs the sum to node 712. Node 712 subtracts the filtered walking, chest signal 718 from the sum provided by node 710 to produce a breathing signal 714, which is fed back to LMS module 720. LMS module 720 and filter 716 operate based on the equations shown at 724.

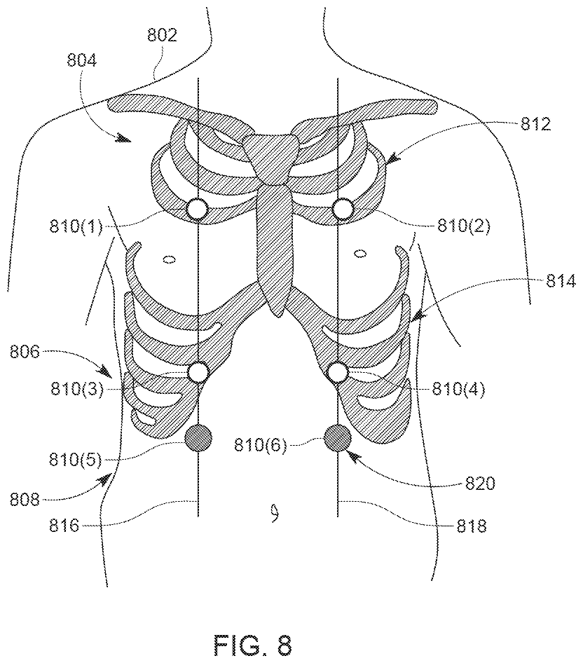

To accommodate the low sensitivities at very low frequencies, a pair of accelerometers for each axis and differential readings between the pair may be utilized to improve accuracy. FIG. 8 is a diagram illustrating an arrangement of IMUs 810(1)-810(6) on a human subject 802 to accommodate low sensitivities at very low frequencies, and to detect differential movement of the thoracic ribcage, abdominal ribcage, and abdomen. As shown in FIG. 8, pairs of IMUs 810(1)-810(6) are positioned on the chest and the abdomen to enable differences between pairs to improve accuracy at low frequencies. Specifically, the laterally spaced IMUs 810(1) and 810(2) on the third ribs 812 enable more accurate lateral displacement estimation on the upper chest 804, and the laterally spaced IMUs 810(3) and 810(4) on the eight ribs 814 enable more accurate lateral displacement estimation on the lower chest 806. The pair of laterally spaced IMUs 810(5) and 810(6) on the abdomen 808 enable more accurate lateral abdominal displacements. Since the sensors on the two lateral sides of the chest/abdomen experience opposite lateral displacements, using the difference between sensor signals increases signal amplitude and improves accuracy for low-frequency low-displacement measurements. In an example, IMUs 810(1), 810(3), and 810(5) are vertically aligned with each other along a vertical line 816 that goes through the medial one third of one clavicle, and IMUs 810(2), 810(4), and 810(6) are vertically aligned with each other along a vertical line 818 that goes through the medial one third of the other clavicle. In an example, IMUs 810(5) and 810(6) are positioned at a midpoint between the xiphoid process and the umbilicus.

Some examples disclosed herein perform a method for computing a phase difference between chest and abdominal motions. The phase difference between the displacements measured on two or three different wearable straps (e.g., straps on the chest and the abdomen of the subject) or adhesive pads may be estimated. To estimate the phase difference, two displacements along the same axes (either the x, y or z axes) measured on two straps (or adhesive pads) are utilized as follows: (1) The cross correlation between any two corresponding displacements is found in real-time; (2) one of the two displacements is delayed by N time-steps and then the cross-correlation is determined between this delayed displacement and the other non-delayed displacement; (3) the value of the time delay N is varied from 1 to 20, and the cross correlation is obtained for each of these values of N; (4) the value of N equal to Nₒₚₜ which provides the best cross-correlation is identified as the time delay at which the delayed displacement is best in-sync with the other displacement; and (5) the value NₒₚₜTₛ where Tₛ is the sampling period is then the time delay between the displacements on the two straps (or adhesive pads).

Some examples disclosed herein involve the detection of thoracoabdominal asynchrony. In normal breathing, the displacements of the rib cage and abdomen are generally in phase, meaning outward and inward motions occur in near-synchrony. The presence of relative phase lag between the two compartments is referred to as thoracoabdominal asynchrony (TAA). Paradoxical breathing is the extreme case of TAA and is characterized by compartmental displacements that are fully out of phase during respiration. The presence of TAA and/or paradoxical breathing is associated with respiratory distress, as in COPD.

To detect and estimate TAA, some examples utilize the cross correlation extracted from a moving window of the anteroposterior displacements. Cross correlation has previously been shown to be the best quantitative estimator of TAA phase angle in the context of experimental RIP. Defining the window size to be samples long, then the window of anteroposterior displacements from IMU p at time step k is:

x_{y}(k)=[xₖ(k)−xₖ(k+t−1)]T Equation III

Since the abdominal and chest displacement magnitudes may not be in the same range, we first normalize the displacements such that xₚ is zero mean and xₚ^{T}xₚ=1. The estimate of the cross correlation of IMUs p and q is then given by:

R_{pq}(m)=xₚ(k+m)^{T}x_{q}(k) Equation IV

For simple detection of paradoxical breathing, only R_{pq}(0)(m=0) needs to be calculated, as in-phase displacements have positive cross correlation at zero lag, and out-of-phase displacements have negative cross correlation at zero lag. Thus, a simple threshold may be used to detect paradoxical breathing using R_{pq}(U).

For quantitative estimation of the phase angle, R_{pq} should be calculated across a range of delays, m. The delay mₚₑₐₖ corresponding to the peak cross correlation is then an estimate of the relative lag. The phase angle can then be estimated using the respiratory rate:

\hat{ϕ}=2πmₚₑₐₖf₀T Equation V

where f₀ is the respiratory rate in Hz, and T is the IMU sample period in seconds, and \hat{ϕ} is in radians.

Asynchrony is also commonly presented using Lissajous curves, which plot displacements of the rib cage on the vertical axis against corresponding abdominal displacements. For normal in-phase breathing, the Lissajous curve appears close to a positive slope line segment. For paradoxical breathing, the curve appears as a negative slope line segment. For intermediate phase angles, the curve appears as an ellipse. Lissajous curves are best used qualitatively, as cross correlation techniques are less sensitive to noise and non-sinusoidal breathing patterns.

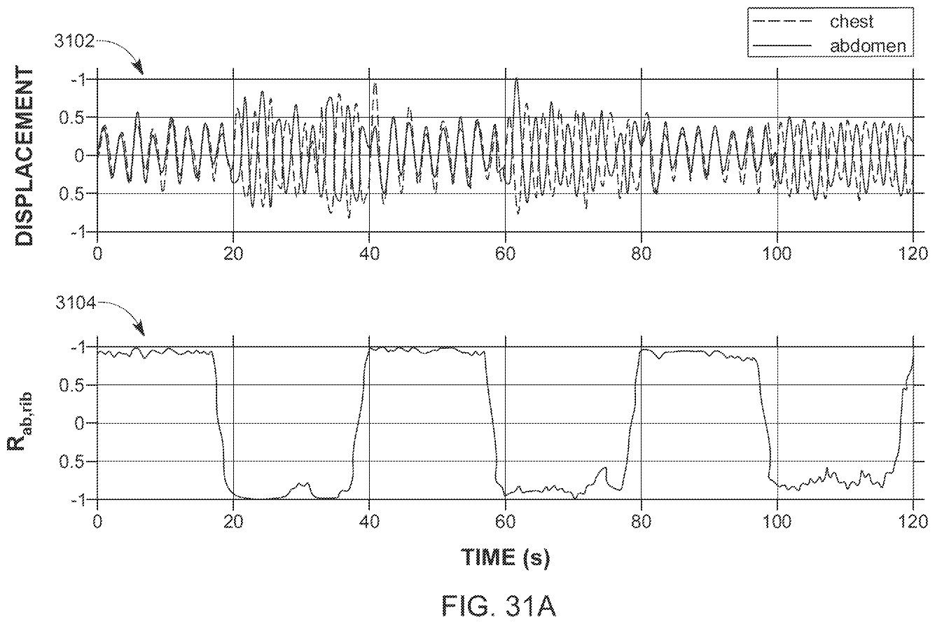

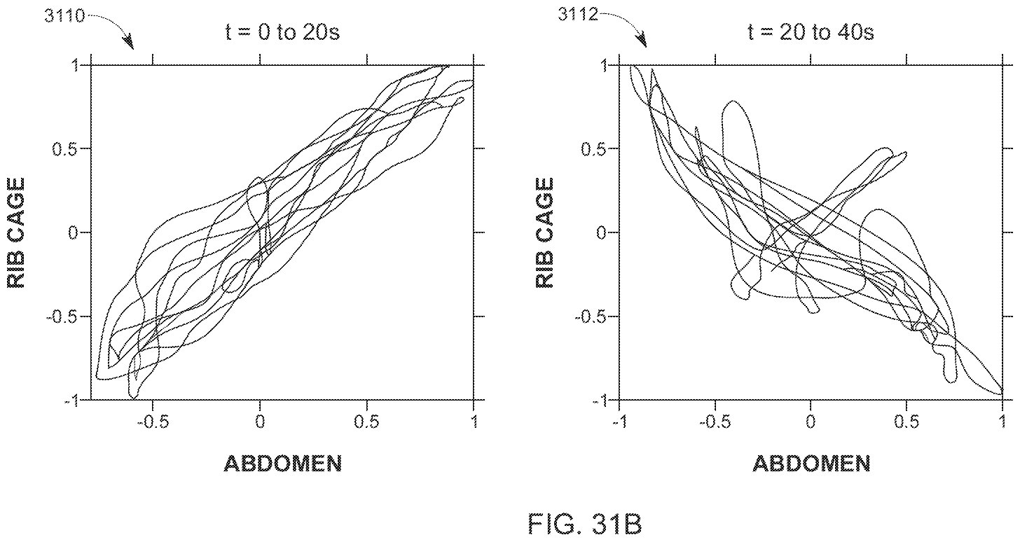

FIGS. 31A and 31B are diagrams illustrating experimental results for detection of paradoxical thoracoabdominal displacements. Every 20 seconds, breath changes from normal (e.g., 0 to 20 s) to breath held while moving abdomen in and out (e.g., 20 to 40 s). Graph 3102 in FIG. 31A shows anteroposterior IMU-based displacement estimates, and graph 3104 in FIG. 31A shows the corresponding cross correlation, which changes from approximately +1 to −1 when motion changes from normal to paradoxical. FIG. 31B shows the Lissajous curve 3110 for the first twenty second interval, and the Lissajous curve 3112 for the second twenty second interval.

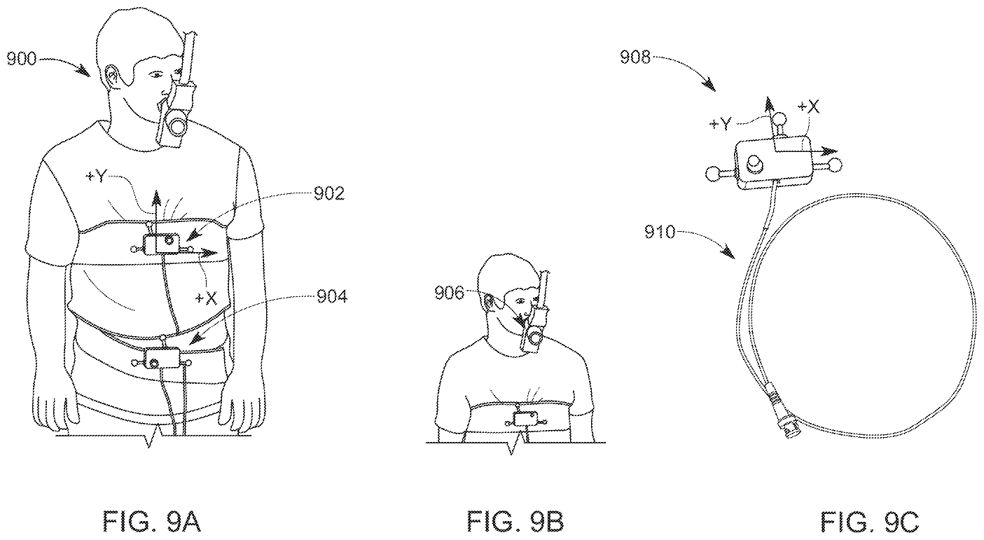

FIGS. 9A-9C are diagrams illustrating one example implementation of a system 900 with wearable ambulatory sensors for measurement of breathing motions. As shown in FIGS. 9A-9C, the system 900 includes a chest strap 902 with an IMU 908, an abdominal strap 904 with an IMU 908, a spirometer mouthpiece 906, and an OptiTrack sync cable 910. Straps 902 and 904 were used in this instance to secure both IMU and OptiTrack device. In other examples, adhesive pads may be used in place of straps 902 and 904. In the illustrated example, each of the IMUs 908 is a 6-axis Invensense ICM42605 IMU powered by a Li-ion rechargeable battery (1000 mAH). IMU data is recorded at 1000 Hz to an on board microSD card, and is synchronized with an optiTrack system using a triggering system. Gravity is estimated and removed from accelerometer using an error-state Kalman smoother with gyro bias estimation. KF parameters are determined using experiments. Accelerometer data is double integrated and low pass filtered.

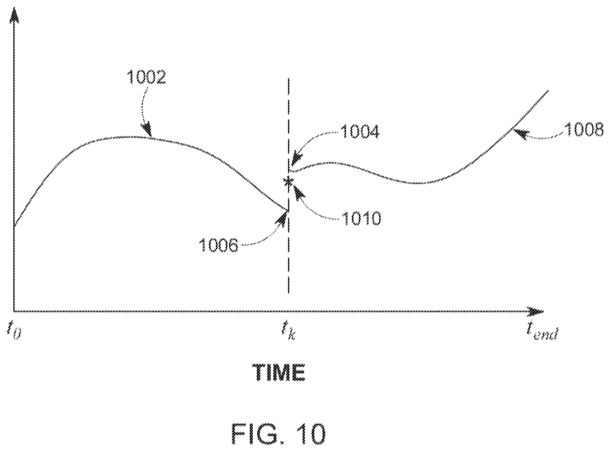

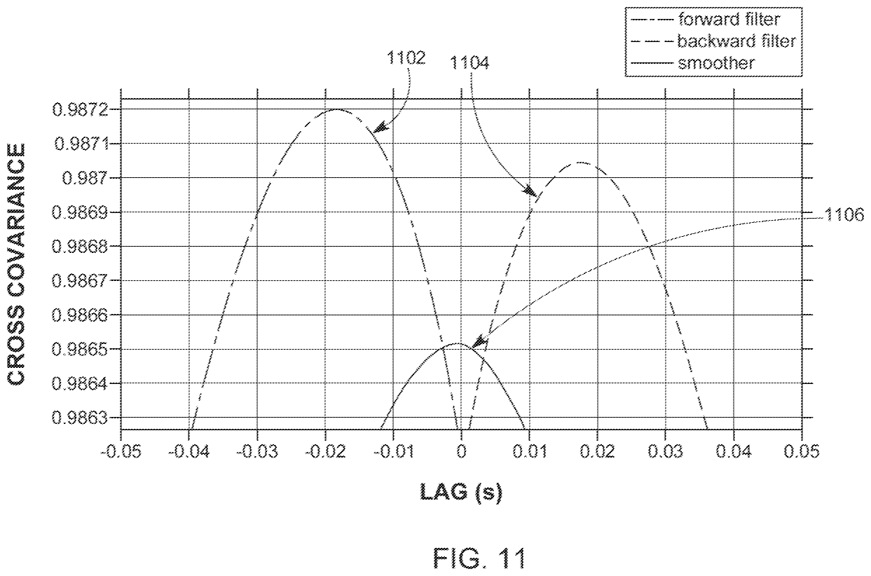

FIG. 10 is a diagram illustrating a Kalman smoother overview according to one example. FIG. 10 shows forward Kalman filter 1002, backward estimate at tₖ 1004, forward estimate at tₖ 1006, backward Kalman filter 1008, and smoother estimate 1010 found by weighted combination of forward and backward estimates. FIG. 11 is a diagram illustrating how the Kalman smoother solves the lag issue. FIG. 11 shows forward filter curve 1102, backward filter curve 1104, and smoother curve 1106. The forward and backward filters have lags in opposite time directions, and the smoother has no lag.

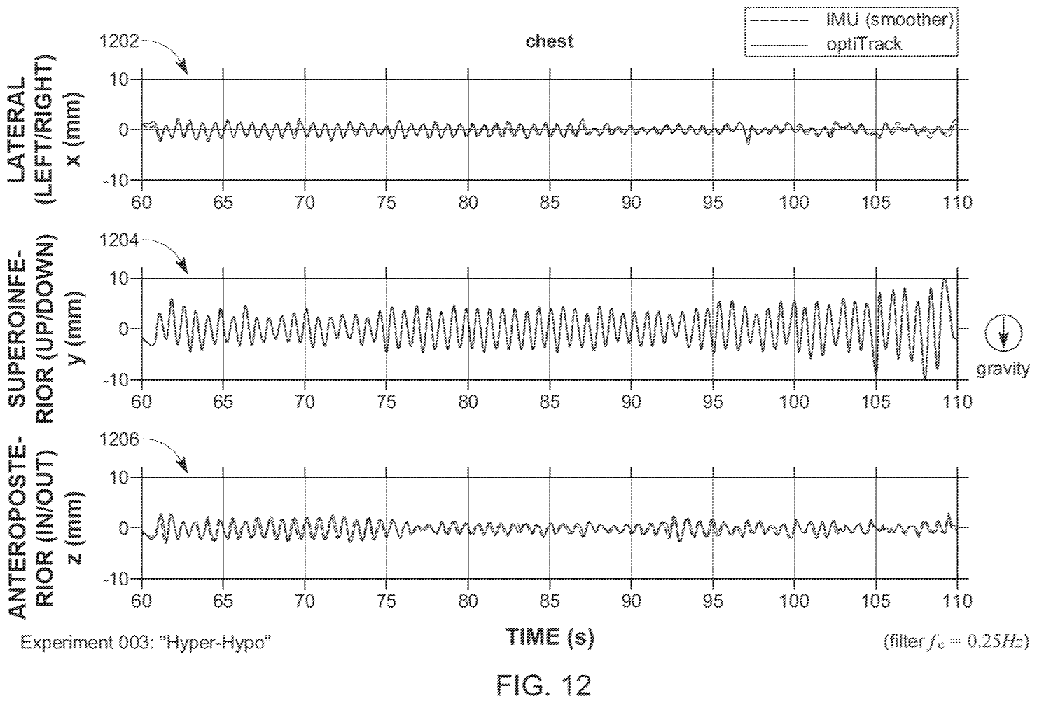

FIGS. 12-26 are diagrams illustrating graphs of measurements made by the system shown in FIG. 9. FIG. 12 shows graphs of chest measurements made while the user is sitting and hyperventilating (f₀=1.4 Hz). In FIG. 12, graph 1202 represents signals along the x-axis, graph 1204 represents signals along the y-axis, and graph 1206 represents signals along the z-axis. It can be seen that the IMU signals accurately match the measurements by the infrared optiTrack camera system.

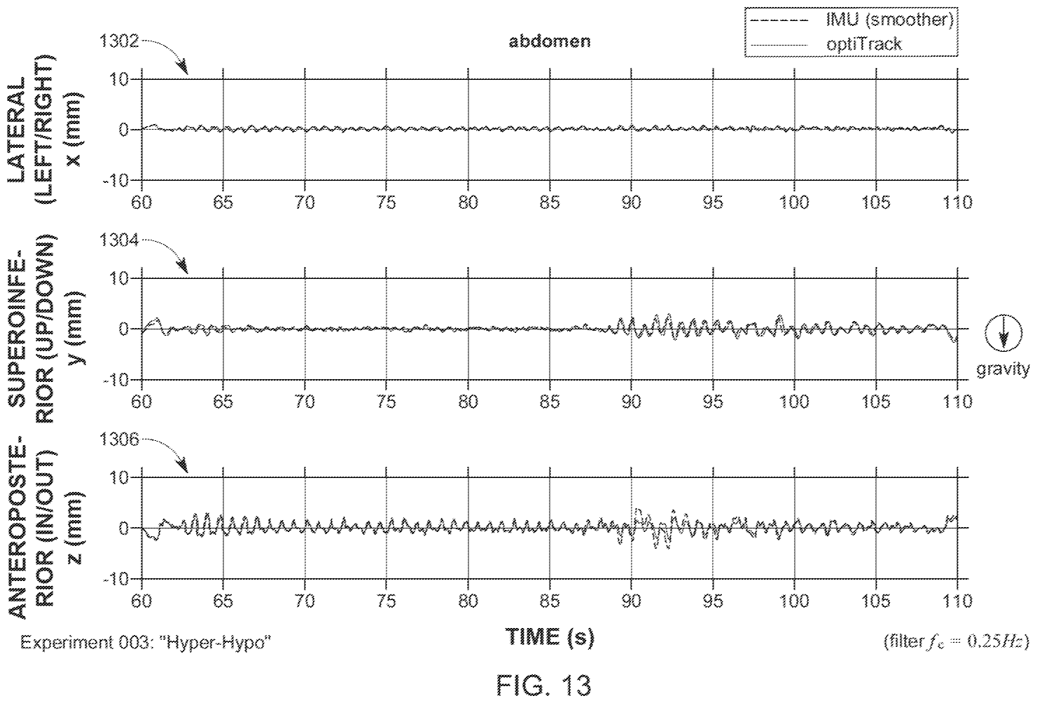

FIG. 13 shows graphs of abdomen measurements made while the user is sitting and hyperventilating (f₀=1.4 Hz). In FIG. 13, graph 1302 represents signals along the x-axis, graph 1304 represents signals along the y-axis, and graph 1306 represents signals along the z-axis. It can be seen that the IMU signals accurately match the measurements by the infrared optiTrack camera system.

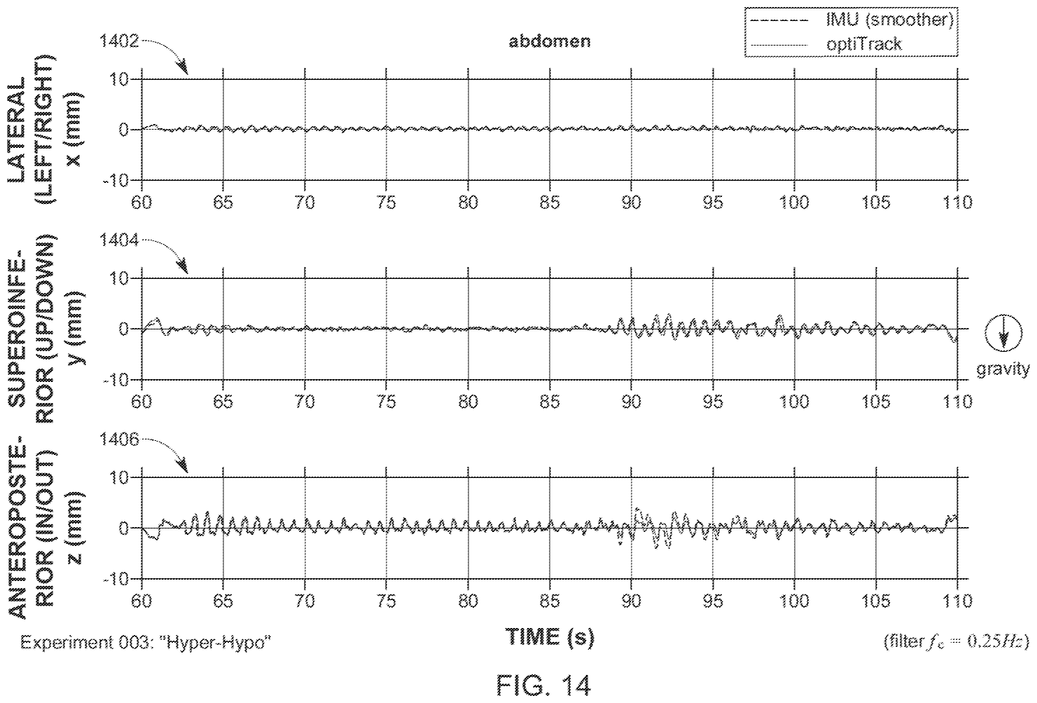

FIG. 14 shows graphs of abdomen measurements made while the user is sitting (f₀=1.4 Hz). In FIG. 14, graph 1402 represents signals along the x-axis, graph 1404 represents signals along the y-axis, and graph 1406 represents signals along the z-axis. It can be seen that the IMU signals accurately match the measurements by the infrared optiTrack camera system.

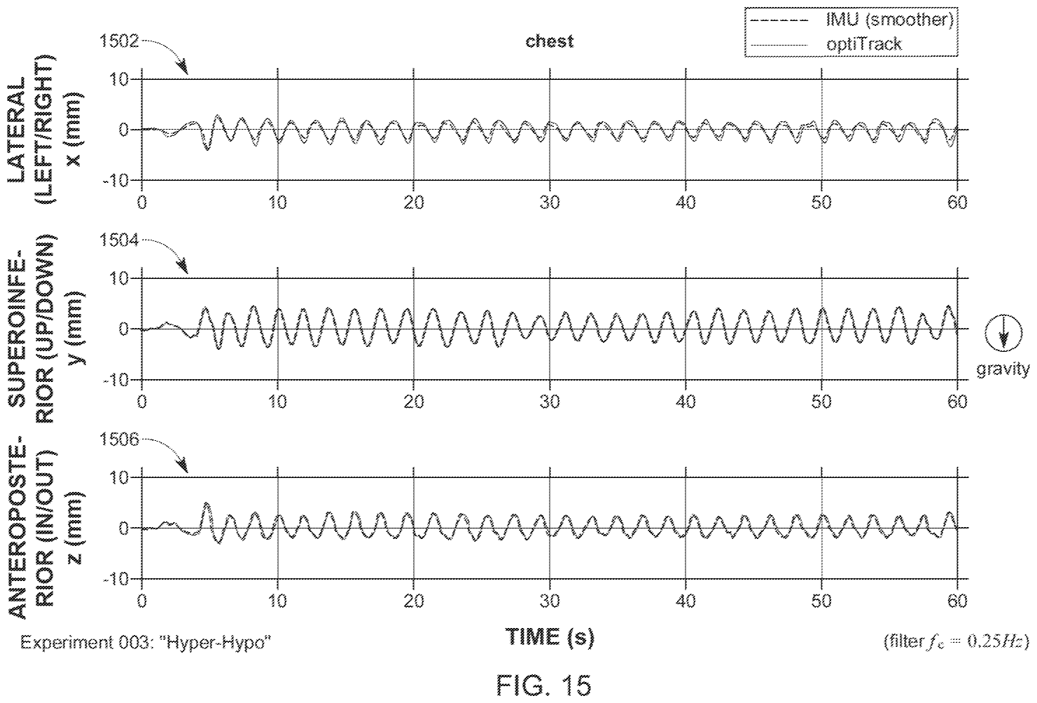

FIG. 15 shows graphs of chest measurements made while the user is sitting and breathing normally (f₀=0.5 Hz). In FIG. 15, graph 1502 represents signals along the x-axis, graph 1504 represents signals along the y-axis, and graph 1506 represents signals along the z-axis. It can be seen that the IMU signals accurately match the measurements by the infrared optiTrack camera system.

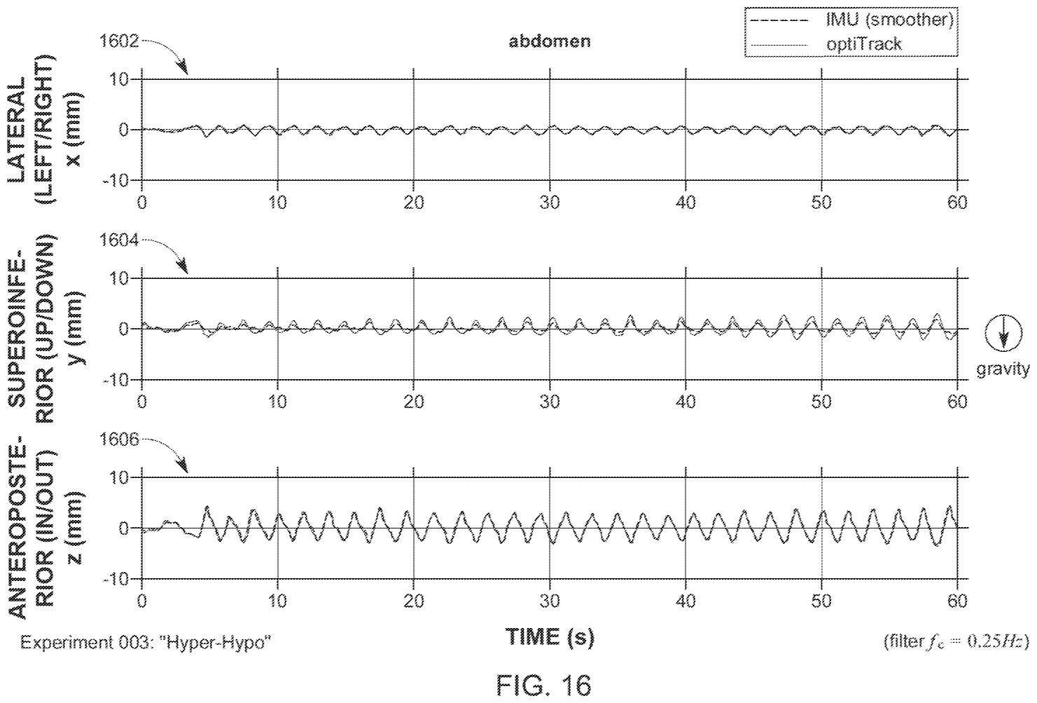

FIG. 16 shows graphs of abdomen measurements made while the user is sitting and breathing normally (f₀=0.5 Hz). In FIG. 16, graph 1602 represents signals along the x-axis, graph 1604 represents signals along the y-axis, and graph 1606 represents signals along the z-axis. It can be seen that the IMU signals accurately match the measurements by the infrared optiTrack camera system.

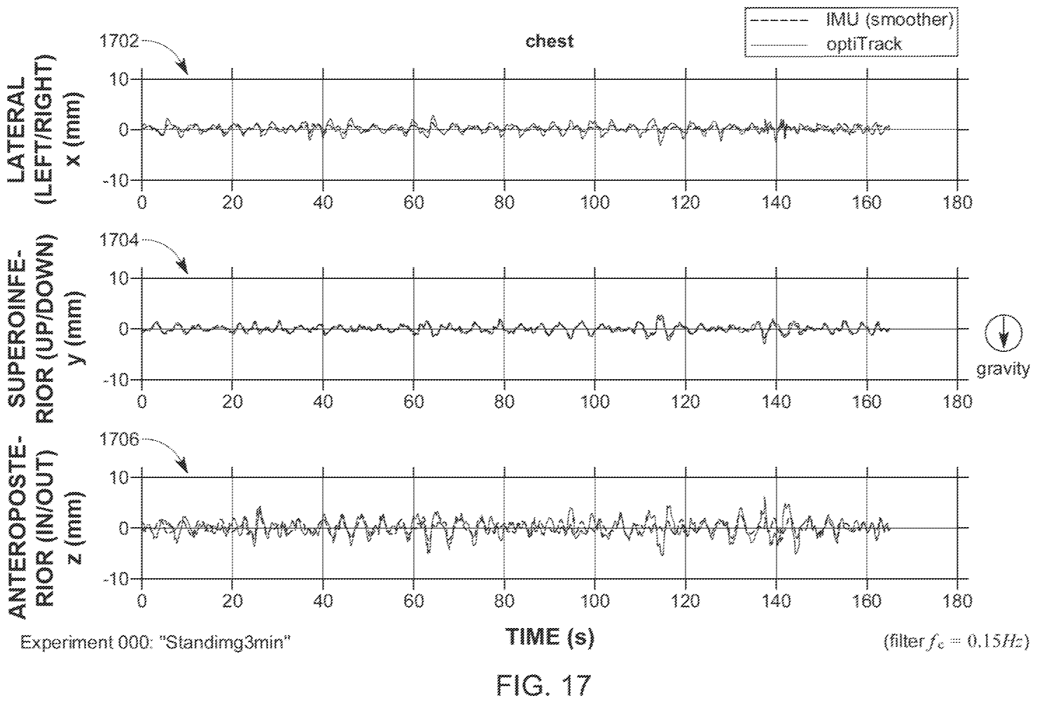

FIG. 17 shows graphs of chest measurements made while the user is freely standing (0.20-0.28 Hz). In FIG. 17, graph 1702 represents signals along the x-axis, graph 1704 represents signals along the y-axis, and graph 1706 represents signals along the z-axis. It can be seen that the IMU signals accurately match the measurements by the infrared optiTrack camera system.

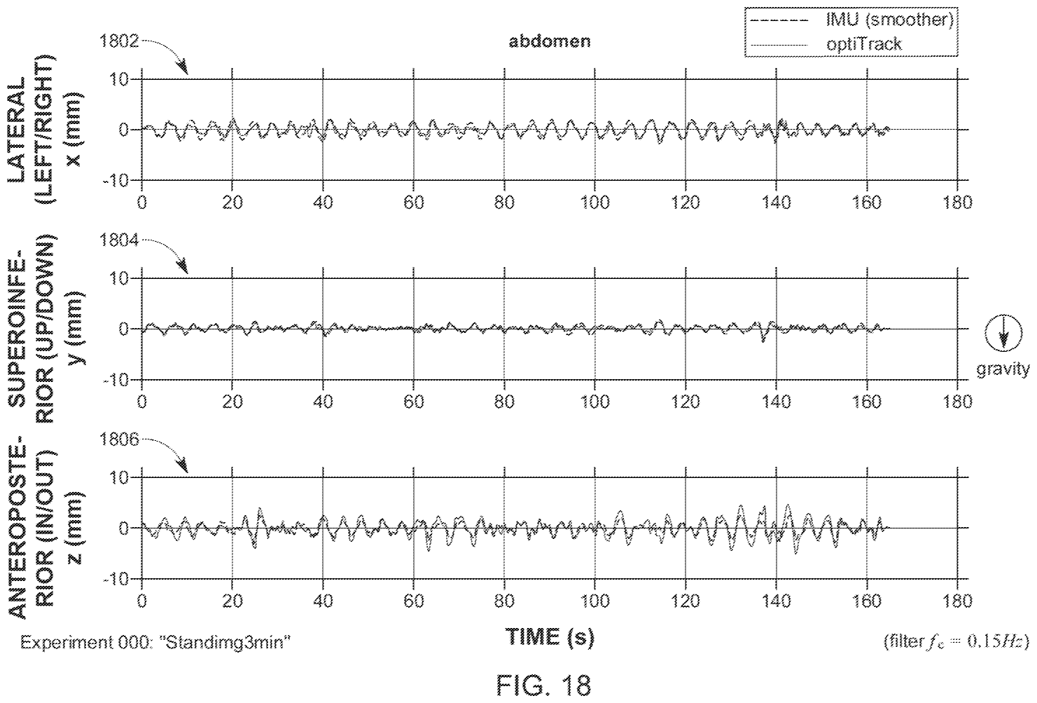

FIG. 18 shows graphs of abdomen measurements made while the user is freely standing (0.20-0.28 Hz). In FIG. 18, graph 1802 represents signals along the x-axis, graph 1804 represents signals along the y-axis, and graph 1806 represents signals along the z-axis. It can be seen that the IMU signals accurately match the measurements by the infrared optiTrack camera system.

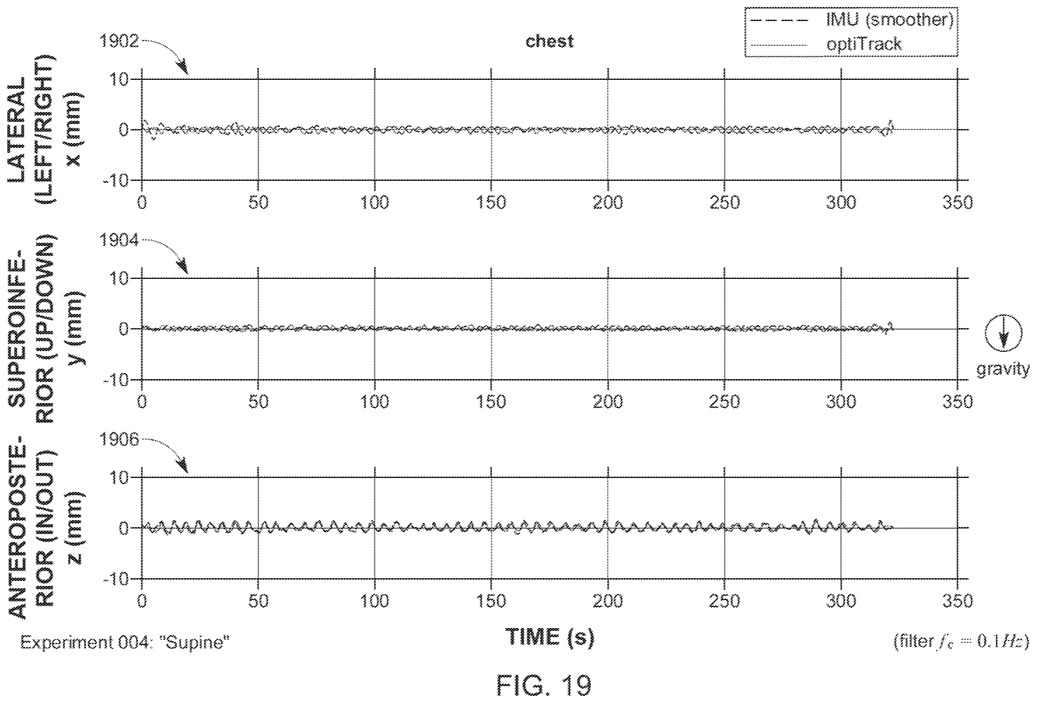

FIG. 19 shows graphs of chest measurements made while the user is in a supine position (0.15-0.20 Hz). In FIG. 19, graph 1902 represents signals along the x-axis, graph 1904 represents signals along the y-axis, and graph 1906 represents signals along the z-axis. It can be seen that the IMU signals accurately match the measurements by the infrared optiTrack camera system.

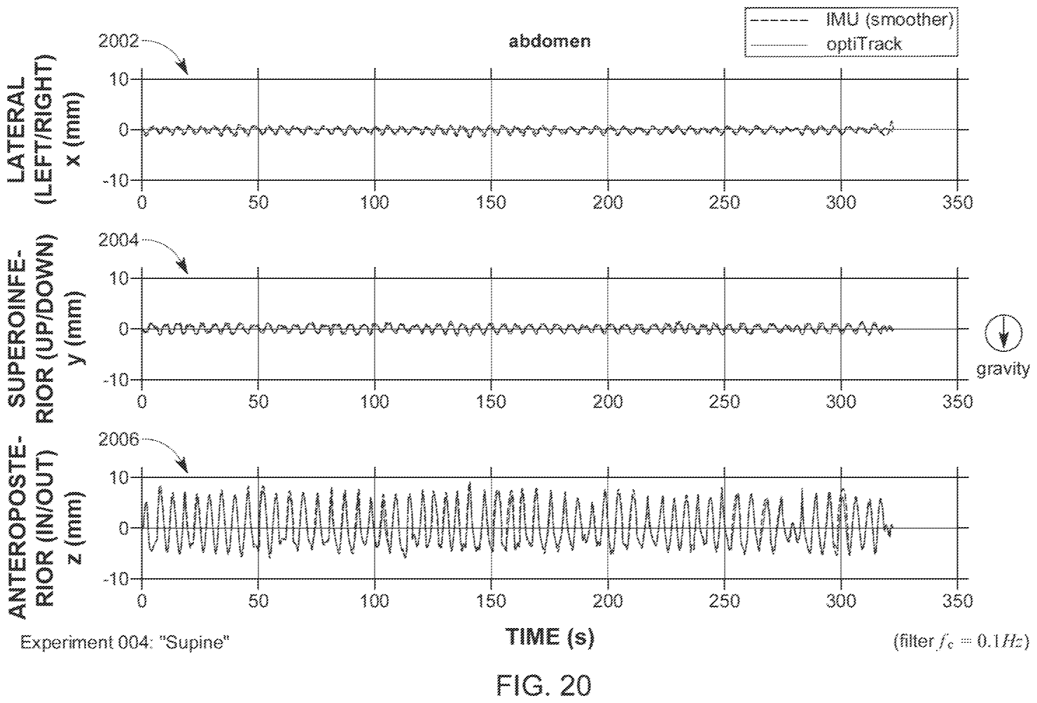

FIG. 20 shows graphs of abdomen measurements made while the user is in a supine position (0.15-0.20 Hz). In FIG. 20, graph 2002 represents signals along the x-axis, graph 2004 represents signals along the y-axis, and graph 2006 represents signals along the z-axis. It can be seen that the IMU signals accurately match the measurements by the infrared optiTrack camera system.

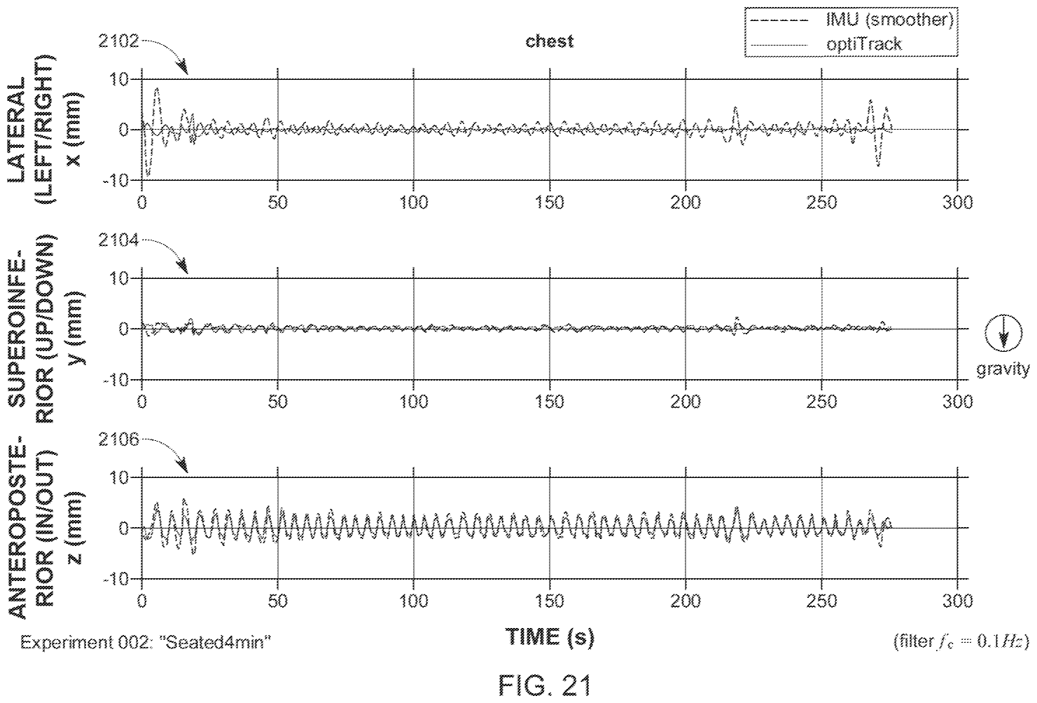

FIG. 21 shows graphs of chest measurements made while the user is sitting (0.15-0.25 Hz). In FIG. 21, graph 2102 represents signals along the x-axis, graph 2104 represents signals along the y-axis, and graph 2106 represents signals along the z-axis. It can be seen that the IMU signals accurately match the measurements by the infrared optiTrack camera system.

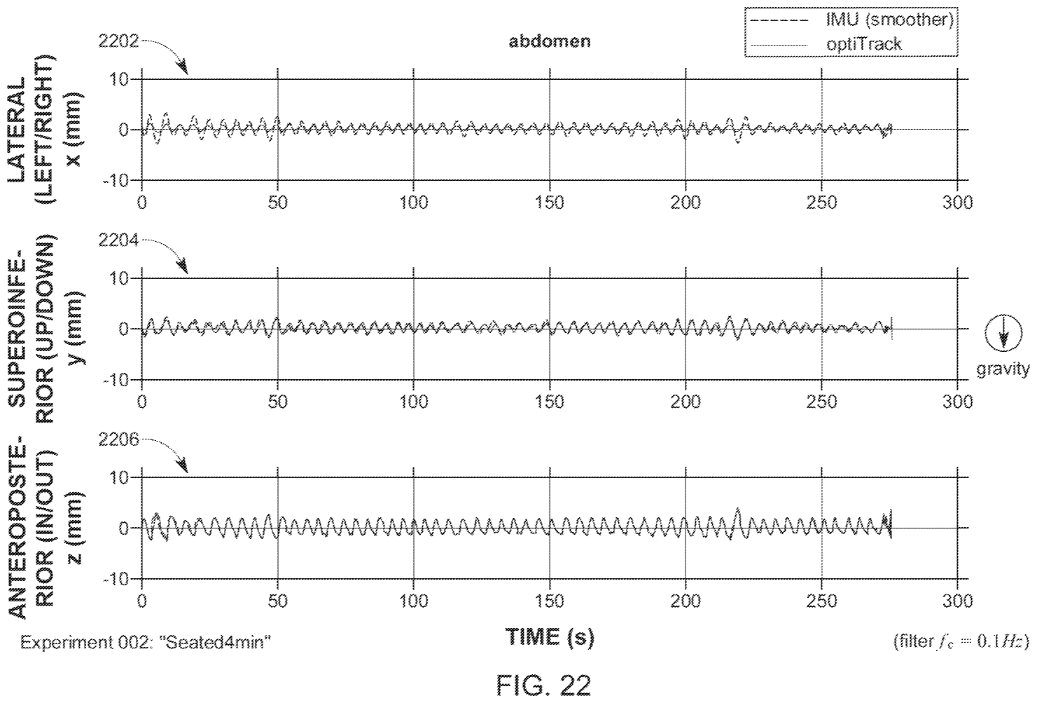

FIG. 22 shows graphs of abdomen measurements made while the user is sitting (0.15-0.25 Hz). In FIG. 22, graph 2202 represents signals along the x-axis, graph 2204 represents signals along the y-axis, and graph 2206 represents signals along the z-axis. It can be seen that the IMU signals accurately match the measurements by the infrared optiTrack camera system.

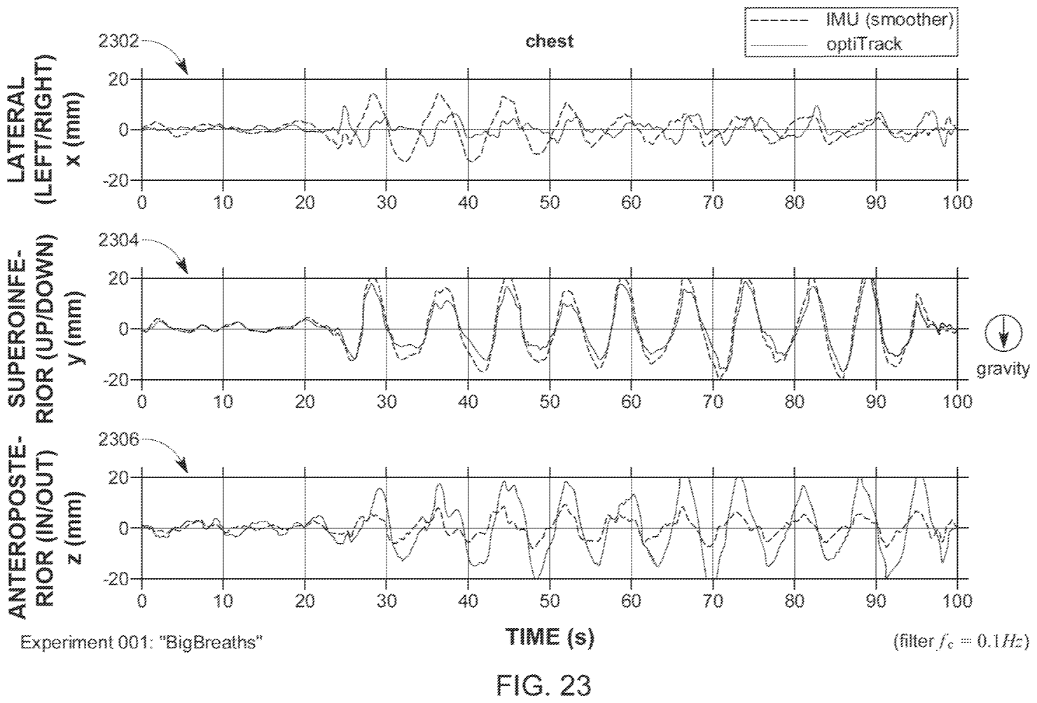

FIG. 23 shows graphs of chest measurements made while the user is freely standing and taking “big breaths” (0.12-0.15 Hz). In FIG. 23, graph 2302 represents signals along the x-axis, graph 2304 represents signals along the y-axis, and graph 2306 represents signals along the z-axis. It can be seen that the IMU signals accurately match the measurements by the infrared optiTrack camera system.

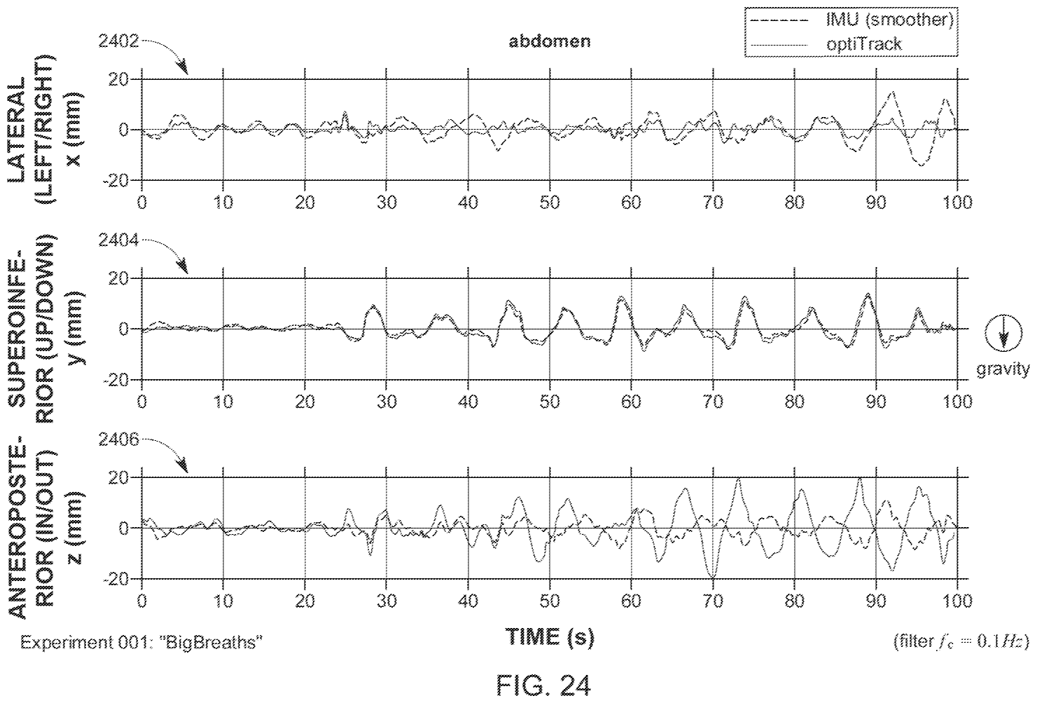

FIG. 24 shows graphs of abdomen measurements made while the user is freely standing and taking “big breaths” (0.12-0.15 Hz). In FIG. 24, graph 2402 represents signals along the x-axis, graph 2404 represents signals along the y-axis, and graph 2406 represents signals along the z-axis. It can be seen that the IMU signals accurately match the measurements by the infrared optiTrack camera system.

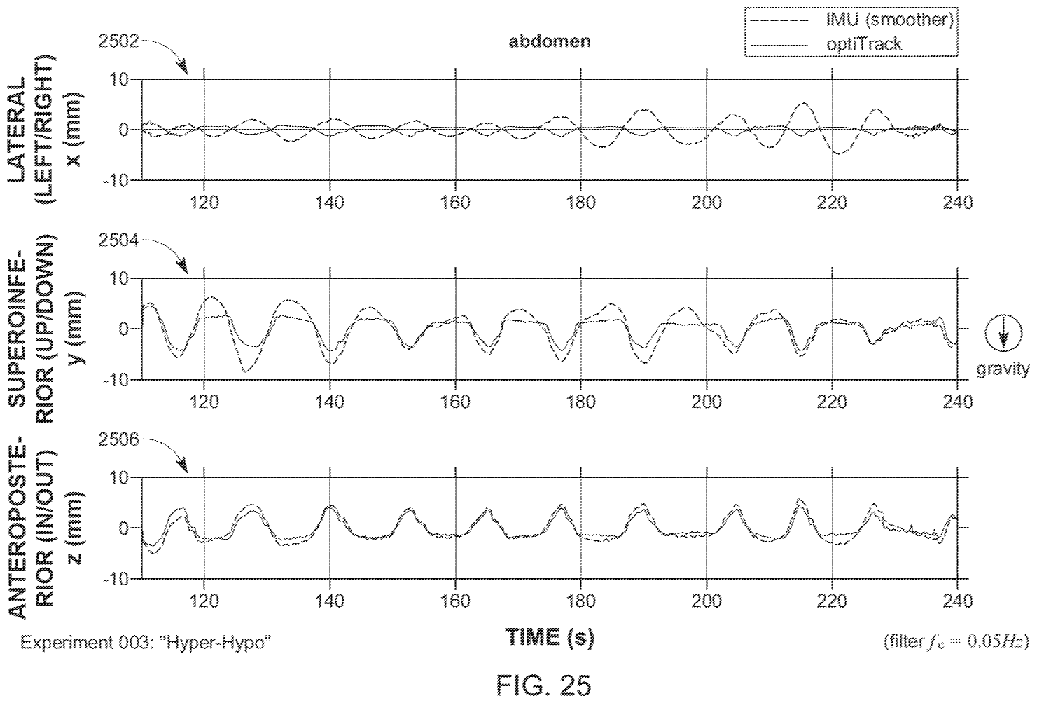

FIG. 25 shows graphs of abdomen measurements made while the user is sitting (f₀=0.08 Hz). In FIG. 25, graph 2502 represents signals along the x-axis, graph 2504 represents signals along the y-axis, and graph 2506 represents signals along the z-axis. It can be seen that the IMU signals accurately match the measurements by the infrared optiTrack camera system.

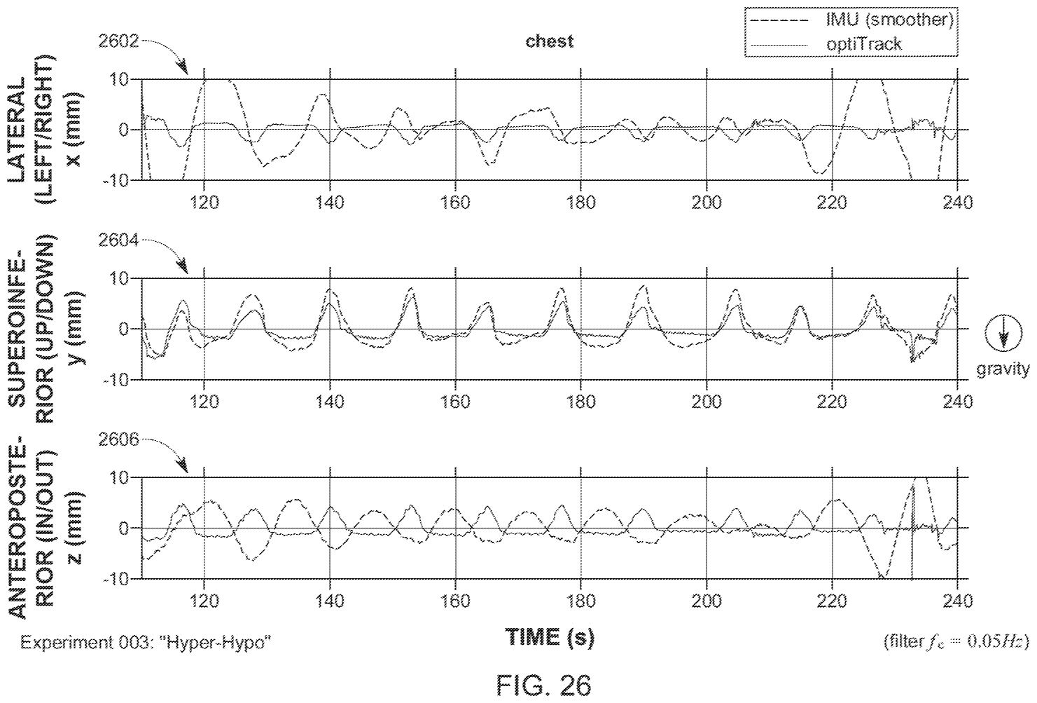

FIG. 26 shows graphs of chest measurements made while the user is sitting (f₀=0.08 Hz). In FIG. 26, graph 2602 represents signals along the x-axis, graph 2604 represents signals along the y-axis, and graph 2606 represents signals along the z-axis. It can be seen that the IMU signals accurately match the measurements by the infrared optiTrack camera system.

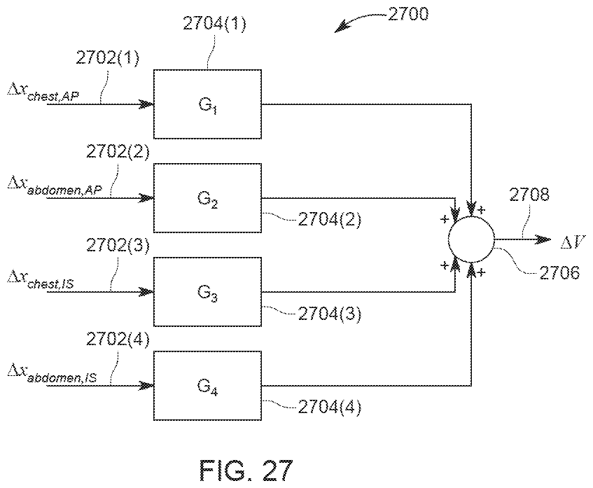

Some examples disclosed herein are configured to estimate both surface displacements and tidal volume. In some examples, a linear transfer function, a finite impulse response (FIR) filter, or an infinite impulse response (IIR) filter may be used in the estimation of tidal volume. Linear transfer functions may be implemented as n-tap FIR filters. FIG. 27 is a diagram illustrating a linear FIR filter model 2700 that may be used to estimate tidal volume according to one example. Variables used in the FIR filter model are defined in the following Table

TABLE I

| Variable | Explanation |
|---|---|
| ΔV | Change in volume |
| Δx_{chest, AP} | Chest displacement, anteroposterior |
| Δx_{abdomen, AP} | Abdomen displacement, anteroposterior |
| Δx_{chest, IS} | Chest displacement, inferosuperior |
| Δx_{abdomen, IS} | Abdomen displacement, inferosuperior |
| C₁, C₂, C₃, etc. | Constants |

As shown in FIG. 27, weights 2704(1)-2704(4) (G₁-G₄) are applied to inputs 2702(1)-2702(4), respectively, and the results are summed at node 2706 to generate the change in volume 2708. In an example, input 2702(1) is chest displacement, anteroposterior; input 2702(2) is abdomen displacement, anteroposterior; input 2702(3) is chest displacement, inferosuperior; and input 2702(4) is abdomen displacement, inferosuperior. The linear FIR filter may be implemented as an n-tap FIR filter, and filter weights can be trained using Multiple Linear Regression on a batch of training data.

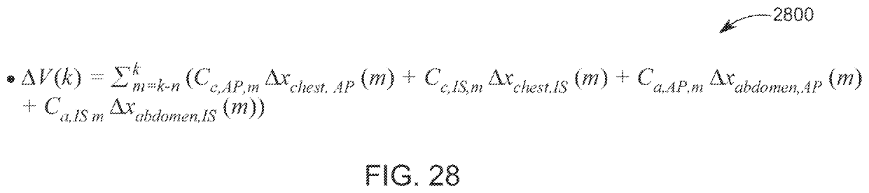

Tidal volume may be modeled using linear n-tap FIR filters on each displacement, as shown in FIG. 28. FIG. 28 is a diagram illustrating an equation 2800 for calculating tidal volume according to one example.

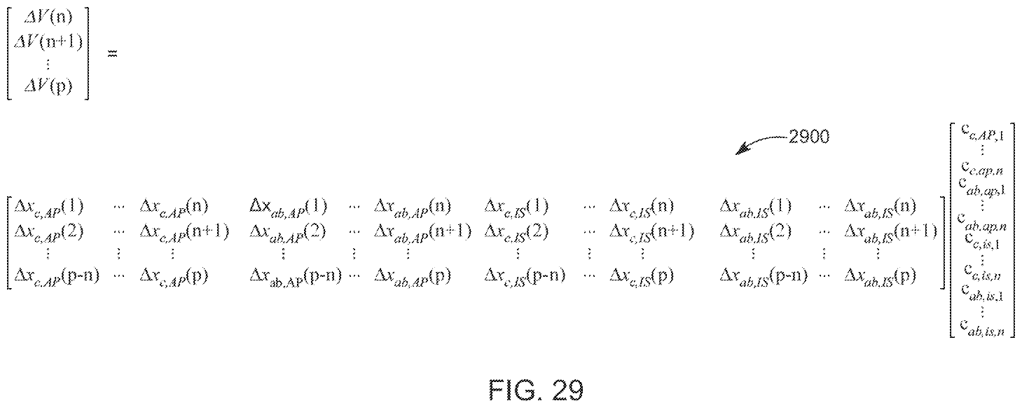

Given a series of spirometer data ΔV(1), ΔV(2), . . . , ΔV(p) along with anteroposterior and inferosuperior displacements Δx_{AP}(1), . . . , Δx_{AP}(p) and Δx_{IS}(1), . . . , Δx_{IS}(p) for each IMU, then the FIR model can be rewritten in vector matrix form V=Xc as shown in the equation 2900 in FIG. 29. FIG. 29 is a diagram illustrating model fitting using a training set batch for the linear FIR filter model shown in FIG. 14 according to one example. For two IMUS and p>5n, the system can solve for optimal parameter vector c in the least squares sense.

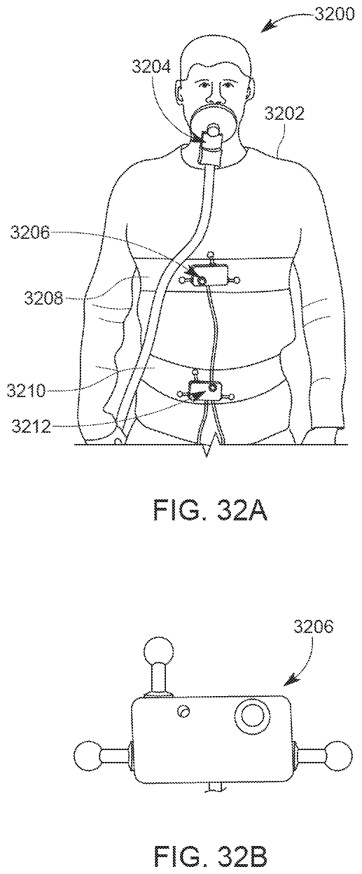

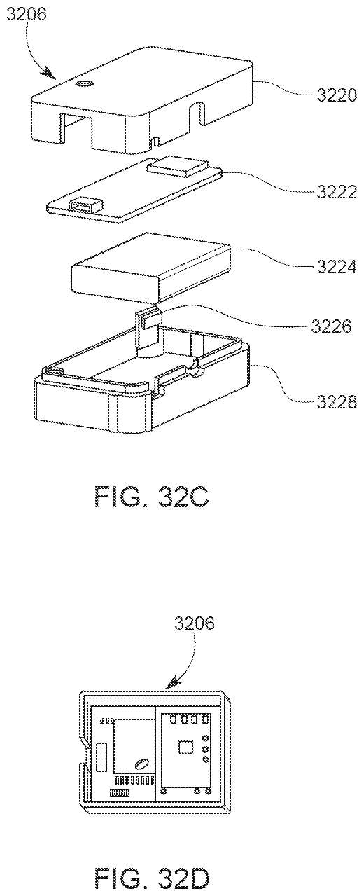

FIGS. 32A-32D are diagrams illustrating a system 3200 for estimating tidal volume according to an example. As shown in FIG. 32A, system 3200 includes a spirometer 3204, an elastic band 3208 around a chest of a user 3202, and an elastic band 3210 around an abdomen of the user 3202. The elastic band 3208 includes a chest sensing unit 3206. The elastic band 3212 includes an abdomen sensing unit 3212. FIG. 32B shows a zoomed-in view of the chest sensing unit 3206, and FIG. 32C shows an assembly view of the chest sensing unit 3206. In an example, abdomen sensing unit 3212 is configured in the same manner as the chest sensing unit 3206. As shown in FIG. 32C, chest sensing unit 3206 includes a top enclosure 3220, a printed circuit board (PCB) with an IMU 3222, a batter 3224, a switch 3226, and a bottom enclosure 3228. FIG. 32D shows the chest sensing unit 3206 with the top enclosure 3220 removed.

An objective of the system 3200 is double integration of corrected acceleration measurements to obtain three-dimensional respiratory displacements. The corrections to the accelerometer's signals compensate for bias and gravity components which have a periodic variation with back-and-forth tilting of the thorax during breathing. From the obtained thoracoabdominal displacements, temporal, phasic, and volumetric respiratory variables can be estimated. Respiratory volume may be calculated as follows:

$$Equation VI$$

$$v ( t ) = {\sum }_{t = 1}^{3} ( k \text{?} x \text{?} ( t ) + k \text{?} x \text{?} ( t ) ) + k \text{?} x \text{?} ( t ) + k \text{?}$$

$$\text{?} \text{indicates text missing or illegible when filed}$$

where:
   v(t)=respiratory volume;
   t=1, 2, . . . ;
   i indicates the axis of the IMU (e.g., anteroposterior, inferosuperior, and mediolateral);

[symbol]

[symbol]

[symbol]

[symbol]

The following Tables II and III show volume estimation for different models. In Tables II and III, the model order indicates which variables were used as regressors and to what order. For instance, a 2 indicates the first and second order terms were allowed in the model. In Tables II and III, AP=anteroposterior displacement; ML=mediolateral displacement; IS=inferosuperior displacement; and RSS=root sum square displacement.

TABLE II

| | | | | | | | | | V_{I} | | | | |
|---|---|---|---|---|---|---|---|---|---|---|---|---|---|
| model order | | | | | | | | | validation | | training | | |
| chest | | | | Abdomen | | | | | RMSE | | RMSE | | Adj. |
| AP | ML | IS | RSS | AP | ML | IS | RSS | t_{Tot} | (L) | NRMSE | (L) | NRMSE | R² |
| 1 | | | | 1 | | | | | 0.132 | 0.048 | 0.131 | 0.047 | 0.937 |
| 1 | 1 | 1 | | 1 | 1 | 1 | | | 0.130 | 0.047 | 0.129 | 0.046 | 0.939 |
| 1 | 1 | 1 | | 1 | 1 | 1 | | 1 | 0.118 | 0.043 | 0.117 | 0.042 | 0.950 |
| 1 | 1 | 1 | 1 | 1 | 1 | 1 | 1 | 1 | 0.117 | 0.042 | 0.117 | 0.042 | 0.950 |
| 1 | | | | 1 | | | | 1 | 0.120 | 0.043 | 0.120 | 0.043 | 0.948 |
| 2 | 2 | 2 | | 2 | 2 | 2 | | 2 | 0.117 | 0.042 | 0.116 | 0.042 | 0.951 |
| 3 | 3 | 3 | | 3 | 3 | 3 | | 3 | 0.117 | 0.042 | 0.113 | 0.041 | 0.953 |
| 4 | 4 | 4 | | 4 | 4 | 4 | | 4 | 0.123 | 0.044 | 0.111 | 0.040 | 0.955 |
| 5 | 5 | 5 | | 5 | 5 | 5 | | 5 | 0.124 | 0.045 | 0.110 | 0.040 | 0.956 |
| 6 | 6 | 6 | | 6 | 6 | 6 | | 6 | 0.184 | 0.066 | 0.109 | 0.039 | 0.956 |

TABLE III

| V_{E} | | | | | V_{T} | | | | |
|---|---|---|---|---|---|---|---|---|---|
| validation | | training | | | validation | | training | | |
| RMSE | | RMSE | | Adj. | RMSE | | RMSE | | Adj. |
| (L) | NRMSE | (L) | NRMSE | R² | (L) | NRMSE | (L) | NRMSE | R² |
| 0.126 | 0.062 | 0.126 | 0.062 | 0.856 | 0.210 | 0.051 | 0.209 | 0.051 | 0.922 |
| 0.124 | 0.061 | 0.123 | 0.060 | 0.863 | 0.203 | 0.049 | 0.201 | 0.049 | 0.928 |
| 0.107 | 0.052 | 0.106 | 0.052 | 0.898 | 0.170 | 0.041 | 0.169 | 0.041 | 0.949 |
| 0.106 | 0.052 | 0.105 | 0.052 | 0.900 | 0.170 | 0.041 | 0.168 | 0.041 | 0.950 |
| 0.109 | 0.054 | 0.109 | 0.053 | 0.893 | 0.174 | 0.042 | 0.174 | 0.042 | 0.946 |
| 0.106 | 0.052 | 0.103 | 0.051 | 0.903 | 0.167 | 0.041 | 0.161 | 0.039 | 0.954 |
| 0.105 | 0.051 | 0.102 | 0.050 | 0.906 | 0.173 | 0.042 | 0.158 | 0.038 | 0.956 |
| 0.107 | 0.053 | 0.100 | 0.049 | 0.909 | 0.171 | 0.042 | 0.152 | 0.037 | 0.959 |
| 0.135 | 0.067 | 0.099 | 0.049 | 0.911 | 0.236 | 0.057 | 0.149 | 0.036 | 0.960 |
| 0.168 | 0.082 | 0.099 | 0.049 | 0.912 | 0.645 | 0.157 | 0.147 | 0.036 | 0.961 |

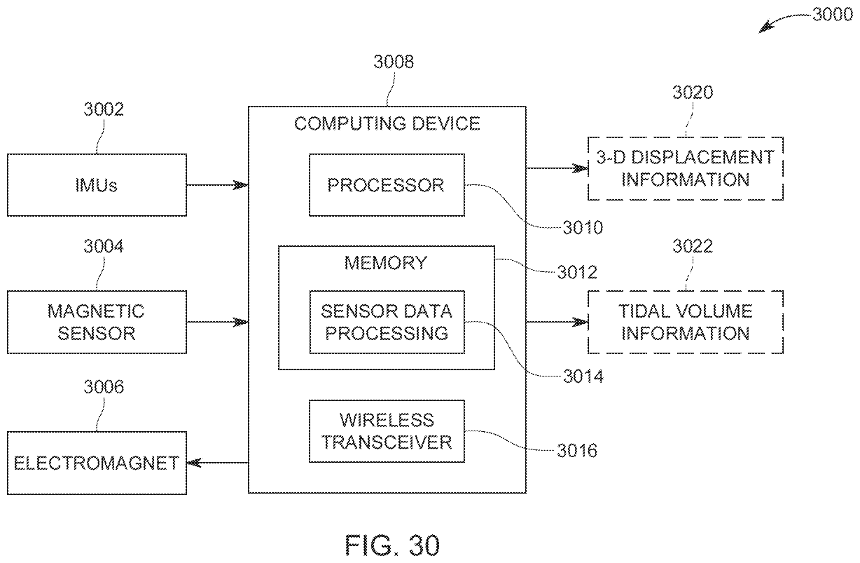

FIG. 30 is a block diagram illustrating a wearable respiratory motion sensor system 3000 according to one example. System 3000 includes a plurality of inertial measurement units (IMUs) 3002, a magnetic sensor 104, an electromagnet 3006, and a computing device 3008. The computing device 3008 includes a processor 3010, a memory 3012, and a wireless transceiver 3016. Memory 3012 stores sensor data processing module 112.

Depending on the exact configuration and type of computing device, the memory 3012 may be volatile (such as RAM), non-volatile (such as ROM, flash memory, etc.), or some combination of the two. The memory 3012 used by computing device 3008 is an example of computer storage media (e.g., non-transitory computer-readable storage media storing computer-executable instructions for performing a method). Computer storage media used by computing device 3008 according to one example includes volatile and nonvolatile, removable and non-removable media implemented in any suitable method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium that can be used to store the desired information and that can be accessed by processor 3010.

In some examples, system 3000 utilizes IMUs 102 to provide sensor information for estimation of chest and abdomen displacements of a human subject. Each IMU 3002 may be implemented as an IMU sensor chip that includes a 3-axis accelerometer and a 3-axis gyroscope. In some examples, the IMUs 3002 may also each include a 3-axis magnetometer. Thus, each IMU 3002 provides either six or nine measurement signals (i.e., three accelerations from the three accelerometers, three rotational rates from the three gyroscopes, and, in some examples, three magnetic field intensities from the three magnetometers).

IMUs 3002 and magnetic sensor 3004 output sensor information to computing device 3008. Processor 3010 executes sensor data processing module 3014 to perform a displacement estimation method and a tidal volume estimation method, as well as other methods disclosed herein, using the received sensor information. Sensor data processing module 3014 outputs three-dimensional (3-D) displacement information 3020 and tidal volume information 3022, based on the processing of the received sensor information.

Computing device 3008 provides an oscillatory current to the electromagnet 3006 to cause the electromagnet 3006 to produce a corresponding oscillatory magnetic field. The magnetic sensor 3004 is used to measure the respiratory frequency of the human subject in real-time by measuring the frequency of variation of the oscillatory magnetic field amplitude generated by electromagnet 3006. The measured respiratory frequency may be used to extract displacements that correspond only to respiratory motion, and not other motions of the human subject.

Wireless transceiver 3016 may be used to wirelessly transmit the 3-D displacement information 3020, tidal volume information 3022, and other information, to a monitoring station for monitoring the human subject. The wearable function and wireless data transmission feature of system 3000 enable inexpensive respiration monitoring in real-world situations over time in ambulatory subjects. System 3000 facilitates earlier diagnosis and intervention for respiratory failure in pediatric patients with neuromuscular disorders. System 3000 may also be used to monitor patient response to interventions to evaluate their effectiveness.

Some examples disclosed herein may be used, for example, in neuromuscular diagnosis and treatment applications for children and adults. Neuromuscular disease with respiratory muscle involvement eventually resulting in respiratory failure is an application with great clinical and commercial impact. It has been recognized for decades that as respiratory muscle weakness progresses, changes in breathing pattern become discernible. Children with neuromuscular disorders generally display abnormal thoracoabdominal patterns of breathing characterized by the asynchrony of the rib cage and the abdominal displacements as well as a diminished contribution of either compartment to tidal volume. This asynchrony is characterized by the phase angle between abdominal and thoracic displacement (normal <20°).

Work in adults with amyotrophic lateral sclerosis (ALS) shows that one can track progression (or improvement, when treatment available) in respiratory muscle performance with appropriate technology. Optoelectronic plethysmography was employed in a population of adults with ALS in whom diaphragm function was relatively preserved. Despite this, the percent contribution of abdominal displacement to tidal volume was significantly lower in ALS patients (53±25%) compared to healthy controls (76±15%, p<0.001) in the supine position. The supine posture—such as one assumes during sleep—poses an additional load on the respiratory muscles, in particular the diaphragm. Abdominal viscera push upward into the chest, resulting in additional diaphragmatic work to generate a tidal breath. As a result, vital capacity falls slightly in the horizontal position. The same technique has been used to monitor thoracoabdominal movement in ALS patients, and it was found that thoracoabdominal asynchrony and paradoxical movement (phase angle 180°) in the middle stage ALS indicates diaphragm impairment and increased thoracic muscle recruitment as compensation. This led to the conclusion that not only resting lung function (e.g., vital capacity) but dynamic chest wall measurements should be taken into account when monitoring disease progression. These preliminary studies provide an indication of the clinical potential of the examples disclosed herein. Moreover, the ambulatory capabilities of the disclosed examples is important to monitor respiratory muscle performance during activities of daily living in vivo and in situ, rather than in a laboratory.

Some examples of the present disclosure may be used for patient-triggered ventilator assistance. Patient-triggered ventilation is the development of modes that proportionally adjust the ventilator inflation pressure in response to the breathing effort of the patient. To provide this ventilator assistance, the ventilator needs a reliable signal from the patient representing the breathing effort. Three ways that may be used to provide such a signal are: (1) airflow changes, used for proportional assist ventilation; (2) diaphragmatic electrical activity used in neurally adjusted ventilatory assistance; and (3) external devices, such as computerized Graseby capsules or plethysmographs detecting the diaphragmatic contractions as abdominal movements and/or thoracic movements. Such external devices generate comparable breathing curves as airflow integrated tidal volumes and transcutaneous or esophageal electromyography of the diaphragm. Quantitation of abdominothoracic movement using phase angle measured by an IMU as disclosed herein may also be used to trigger a breath, rather than simple displacement of each compartment.

Some examples are directed to a wearable breathing sensor system that measures chest wall kinematics during breathing. In particular, the wearable system may measure forward motion of the chest wall, circumferential motion in the horizontal plane, and upward motion of the chest wall (including upper abdomen) along the vertical axis during each respiratory cycle. Time-domain relationships among movement of the abdomen, lower thorax, and upper thorax, may be measured to infer how activation of muscles responsible for movement of these structures are altered during disease evolution. These measurements are useful in providing an accurate assessment of respiratory function for pediatric patients with neuromuscular disorders. The ability to more accurately measure and monitor respiratory muscle function in pediatric patients with neuromuscular disorders enables earlier, targeted, potentially life-saving intervention. By analogy, this method can be used to track improvement in respiratory muscle function in response to emerging genetic and molecular curative therapies. Examples disclosed herein may also be used as an outcome measure to judge the benefits of treatments, such as genetic and molecular treatments in clinical trials in neuromuscular diseases such as Duchenne muscular dystrophy and spinal muscular atrophy.

One example is directed to a wearable respiratory motion sensor system, which includes a plurality of inertial measurement units (IMUs) to be positioned on a subject and generate accelerometer and gyroscope signals. The system also includes a processor to compute three-dimensional displacements of a rib cage and an abdomen of the subject based on the generated accelerometer and gyroscope signals.

The IMUs may be fixed directly to the rib cage and the abdomen of the subject via adhesives, or alternatively, the IMUs may be fixed to at least one wearable strap. The at least one wearable strap may include a first wearable strap (or adhesive pad) configured to be worn around the rib cage of the subject, and a second wearable strap (or adhesive pad) configured to be worn around the abdomen of the subject. In the case of 3 degrees of freedom measurements, at least one wearable strap (or adhesive pad) may include a first wearable strap configured to be worn around the upper rib cage of the subject, a second wearable strap (or adhesive pad) configured to be worn around the lower rib cage of the subject, and a third wearable strap (or adhesive pad) configured to be worn around the abdomen of the subject. The processor may use a signal processing method to remove an influence of sensor bias errors and an influence of gravity on the generated accelerometer and gyroscope signals. The signal processing method may include compensation of a varying influence of gravity based on bending motions of the subject. The processor may compute tidal volume and respiratory rate associated with respiration based on the computed three-dimensional displacements. The processor may compute tidal volume based on the computed three-dimensional displacements using one of a transfer function, a finite impulse response (FIR) filter, or an infinite impulse response (IIR) filter.

Another example is directed to a wearable respiratory motion sensor system, which includes at least one measurement unit to measure multi-dimensional displacements of a rib cage and an abdomen of a subject. The system also includes a processor to perform at least one of the following based on the multi-dimensional displacements: infer muscle groups being utilized during breathing by the subject; monitor health of the subject if the subject has neuromuscular disease conditions or acute respiratory insufficiency; identify a type of respiration of the subject; and detect paradoxical breathing of the subject.

The at least one measurement unit may include a plurality of inertial measurement units (IMUs). The processor may identify predominant muscle groups (e.g., diaphragm, intercostal, accessory) being utilized during breathing by the subject or the type of respiration of the subject based on a time-delay between chest and abdominal displacement measurements or based on a phase difference between chest and abdominal displacement measurements indicative of thoracoabdominal asynchrony. The time-delay between chest and abdominal displacements may be calculated by the processor using a cross-correlation method. The processor may compute tidal volume and respiratory rate (and their ratio) associated with respiration based on the computed multi-dimensional displacements.

Another example is directed to a wearable respiratory motion sensor system, which includes a plurality of inertial measurement units (IMUs), wherein a first one of the IMUs is to measure either chest or abdominal respiratory displacements of a subject, and wherein a second one of the IMUs is to measure ambulatory motions of the subject. The system also includes a processor to process data generated by the IMUs, including removing an influence of the ambulatory motions from corrupting estimates of chest or abdominal respiratory displacements due to respiration. The second one of the IMUs for measurement of ambulatory motions may be configured to be located on a hip of the subject. The first one of the IMUs for measurement of chest or abdominal respiratory displacements may be configured to be located respectively on the chest or abdomen. The processor may use an adaptive least mean squares method to facilitate removing the influence of the ambulatory motions. The system may further include a sensor to measure respiration rate of the subject, wherein the processor may use the measured respiration rate as a reference signal to facilitate removing the influence of the ambulatory motions.

Another example is directed to a method, which includes positioning a plurality of inertial measurement units (IMUs) on a subject, and generating IMU data with the plurality of IMUs. The method further includes computing, with a processor, three-dimensional displacements of a rib cage and an abdomen of the subject based on the generated IMU data.

Another example is directed to a method, which includes measuring, with at least one measurement unit, multi-dimensional displacements of a rib cage and an abdomen of a subject; and computing, with a processor, tidal volume based on the measured multi-dimensional displacements using one of a transfer function, a finite impulse response (FIR) filter, or an infinite impulse response (IIR) filter.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. A wearable respiratory motion sensor system, comprising:
a plurality of inertial measurement units (IMUs) to be positioned on a subject and generate accelerometer and gyroscope signals; and
a processor to compute three-dimensional displacements of a rib cage and an abdomen of the subject based on the generated accelerometer and gyroscope signals.

2. The wearable respiratory motion sensor system of claim 1, wherein the IMUs are fixed directly to the rib cage and the abdomen of the subject.

3. The wearable respiratory motion sensor system of claim 1, wherein the IMUs are fixed to at least one wearable strap.

4. The wearable respiratory motion sensor system of claim 3, wherein the at least one wearable strap includes a first wearable strap configured to be worn around the rib cage of the subject, and a second wearable strap configured to be worn around the abdomen of the subject.

5. The wearable respiratory motion sensor system of claim 3, wherein the at least one wearable strap includes a first wearable strap configured to be worn around the upper rib cage of the subject, a second wearable strap configured to be worn around the lower rib cage of the subject, and a third wearable strap configured to be worn around the abdomen of the subject.

6. The wearable respiratory motion sensor system of claim 1, wherein the IMUs are configured to be fixed to the subject via at least one adhesive pad.

7. The wearable respiratory motion sensor system of claim 6, wherein the at least one adhesive pad includes a first adhesive pad configured to fix a first one of the IMUs on the rib cage of the subject, and a second adhesive pad configured to fix a second one of the IMUs on the abdomen of the subject.

8. The wearable respiratory motion sensor system of claim 6, wherein the at least one adhesive pad includes a first adhesive pad configured to fix a first one of the IMUs on the upper rib cage of the subject, a second adhesive pad configured to fix a second one of the IMUs on the lower rib cage of the subject, and a third adhesive pad configured to fix a third one of the IMUs on the abdomen of the subject.

9. The wearable respiratory motion sensor system of claim 1, wherein the processor is to use a signal processing method to remove an influence of sensor bias errors and an influence of gravity on the generated accelerometer and gyroscope signals.

10. The wearable respiratory motion sensor system of claim 9, wherein the signal processing method includes compensation of a varying influence of gravity based on bending motions of the subject.

11. The wearable respiratory motion sensor system of claim 1, wherein the processor is to compute tidal volume and respiratory rate associated with respiration based on the computed three-dimensional displacements.

12. The wearable respiratory motion sensor system of claim 1, wherein the processor is to compute tidal volume based on the computed three-dimensional displacements using one of a transfer function, a finite impulse response (FIR) filter, or an infinite impulse response (IIR) filter.

13. A wearable respiratory motion sensor system, comprising:
at least one measurement unit to measure multi-dimensional displacements of a rib cage and an abdomen of a subject; and
a processor to perform at least one of the following based on the multi-dimensional displacements: infer muscle groups being utilized during breathing by the subject; monitor health of the subject if the subject has neuromuscular disease conditions or acute respiratory insufficiency; identify a type of respiration of the subject; and detect paradoxical breathing of the subject.

14. The wearable respiratory motion sensor system of claim 13, wherein the at least one measurement unit comprises a plurality of inertial measurement units (IMUs).

15. The wearable respiratory motion sensor system of claim 13, wherein the processor is to identify predominant muscle groups being utilized during breathing by the subject or the type of respiration of the subject based on a time-delay between chest and abdominal displacement measurements or based on a phase difference between chest and abdominal displacement measurements indicative of thoracoabdominal asynchrony.

16. The wearable respiratory motion sensor system of claim 15, wherein the time-delay between chest and abdominal displacements is calculated by the processor using a cross-correlation method.

17. The wearable respiratory motion sensor system of claim 15, wherein the processor is to compute tidal volume and respiratory rate associated with respiration based on the computed multi-dimensional displacements.

18. A wearable respiratory motion sensor system, comprising:
a plurality of inertial measurement units (IMUs), wherein a first one of the IMUs is to measure either chest or abdominal respiratory displacements of a subject, and wherein a second one of the IMUs is to measure ambulatory motions of the subject; and
a processor to process data generated by the IMUs, including removing an influence of the ambulatory motions from corrupting estimates of chest or abdominal respiratory displacements due to respiration.

19. The wearable respiratory motion sensor system of claim 18, wherein the second one of the IMUs for measurement of ambulatory motions is configured to be located on a hip of the subject.

20. The wearable respiratory motion sensor system of claim 18, wherein the first one of the IMUs for measurement of chest or abdominal respiratory displacements is configured to be located respectively on the chest or abdomen.

21. The wearable respiratory motion sensor system of claim 18, wherein the processor is to use an adaptive least mean squares method to facilitate removing the influence of the ambulatory motions.

22. The wearable respiratory motion sensor system of claim 18, and further comprising:
a sensor to measure respiration rate of the subject; and
wherein the processor is to use the measured respiration rate as a reference signal to facilitate removing the influence of the ambulatory motions.

23. A method, comprising:
positioning a plurality of inertial measurement units (IMUs) on a subject;
generating IMU data with the plurality of IMUs; and
computing, with a processor, three-dimensional displacements of a rib cage and an abdomen of the subject based on the generated IMU data.

24. A method, comprising:
measuring, with at least one measurement unit, multi-dimensional displacements of a rib cage and an abdomen of a subject; and
computing, with a processor, tidal volume based on the measured multi-dimensional displacements using one of a transfer function, a finite impulse response (FIR) filter, or an infinite impulse response (IIR) filter.
